(19) United States
(12) Patent Application Publication
(10) Pub. No.: US 2022/0143215 A1
(43) Pub. Date: May 12, 2022

(54) GENE THERAPY VECTORS FOR TREATMENT OF DANON DISEASE

(71) Applicant: Spacecraft Seven, LLC, Cranbury, NJ (US)

(72) Inventors: Annahita KERAVALA, Cranbury, NJ (US); Raj PRABHAKAR, Cranbury, NJ (US); Gaurav SHAH, Cranbury, NJ (US); Roderick WONG, Cranbury, NJ (US); Naveen YALAMANCHI, Cranbury, NJ (US); Piratip PRATUMSUWAN, Cranbury, NJ (US)

(73) Assignee: Spacecraft Seven, LLC, Cranbury, NJ (US)

(21) Appl. No.: 17/430,107

(22) PCT Filed: Feb. 12, 2020
(86) PCT No.: PCT/US2020/017987

§ 371 Date: Aug. 11, 2021

Related U.S. Application Data
(60) Provisional application No. 62/934,928, filed on Nov. 13, 2019.
(60) Provisional application No. 62/804,521, filed on Feb. 12, 2019.

(51) Int. Cl. A61K 48/00; A61P 21/00; C07K 14/005; C07K 14/705; C12N 15/86

(52) U.S. Cl. CPC A61K 48/005; A61P 21/00; C12N 15/86; C07K 14/70596; C07K 14/005

(57) ABSTRACT

The present disclosure provides gene therapy vectors comprising a polynucleotide sequence encoding a LAMP-2 polypeptide, methods of use thereof, pharmaceutical compositions, and more. In particular, the disclosure provides recombinant AAV vectors having AAVrh74 serotype expressing LAMP-2A, LAMP-2B, or LAMP-2C for use as a therapeutic in, for example, Danon Disease.

### RELATED APPLICATIONS

This application claims priority to U.S. Provisional Patent Appl. No. 62/934,928, filed Nov. 13, 2019, and U.S. Provisional Patent Appl. No. 62/804,521, filed Feb. 12, 2019, each of which is incorporated herein by reference in its entirety.

### SEQUENCE LISTING

This application is being filed electronically via EFS-Web and includes an electronically submitted sequence listing in .txt format. The .txt file contains a sequence listing entitled “ROPA_013_02WO_ST25.txt” created on Feb. 11, 2020 and having a size of ˜61 kilobytes. The sequence listing contained in this .txt file is part of the specification and is incorporated herein by reference in its entirety.

### FIELD OF INVENTION

The invention relates generally to gene therapy for diseases associated with mutations in lysosome-associated membrane protein 2 (LAMP-2, also known as CD107b).

### BACKGROUND

Lysosome-associated membrane protein 2 (LAMP-2, also known as CD107b) is a gene that encodes a lysosome-associated membrane glycoprotein. Alternative splicing of the gene produces three isoforms: LAMP-2A, LAMP-2B, and LAMP-2C. Loss-of-function mutations in LAMP-2 are associated with human diseases, including Danon disease, a familial cardiomyopathy associated with impaired autophagy. Danon disease is a rare but serious cardiac and skeletal myopathy leading to substantial morbidity and early mortality due to arrhythmia and cardiomyopathy. The X-linked nature of inheritance accounts for reported differences in phenotypic severity between men and women. Boucek et al. Genetics in Medicine 13:563-568 (2011). The disease is now understood to be caused by a primary deficiency in lysosome-associated membrane protein-2 (LAMP-2), which functions as a lysosomal membrane receptor in chaperone-mediated autophagy. Nishino et al. Nature 406:906-910 (2000).

### SUMMARY OF THE INVENTION

The present disclosure provides such gene therapy vectors related to LAMP2, methods of use thereof, pharmaceutical compositions, and more. Although clinical use of adeno-associated virus (AAV) vectors is known, the selection of preferred serotype(s) of AAV for gene therapy remains challenging and unpredictable.

The present disclosure provides improved gene therapy vectors comprising a polynucleotide sequence encoding a LAMP-2 polypeptide, methods of use thereof, pharmaceutical compositions, and more. In particular, the disclosure provides recombinant AAV vectors having AAVrh74 serotype expressing LAMP-2A, LAMP-2B, or LAMP-2C for use as a therapeutic in, for example, Danon disease.

Other features and advantages of the invention will be apparent from and encompassed by the following detailed description and claims.

### BRIEF DESCRIPTION OF THE DRAWINGS

FIG. 1 depicts an embodiment of an AAV vector having AAVrh74 serotype.

FIG. 2 shows a bar graph of vector DNA quantification in organs most affected in Danon disease by qPCR.

FIG. 3A shows a bar graph of vector DNA quantification in regions of the heart by qPCR.

FIG. 3B shows a bar graph of vector DNA quantification in muscles by qPCR.

FIG. 4 shows a bar graph of mRNA quantification in organs most affected by Danon disease by RT-qPCR.

FIG. 5A shows a bar graph of mRNA quantification in regions of the heart by RT-qPCR.

FIG. 5B shows a bar graph of mRNA quantification in muscles by RT-qPCR.

FIG. 6A shows a micrograph of semi-quantitative mRNA analysis by RNAscope in an untreated left ventricle.

FIG. 6B shows micrographs of semi-quantitative mRNA analysis by RNAscope in treated left ventricles.

FIG. 7A shows a micrograph of semi-quantitative mRNA analysis by RNAscope in an untreated quadricep.

FIG. 7B shows micrographs of semi-quantitative mRNA analysis by RNAscope in treated quadriceps.

FIG. 8 shows micrographs of semi-quantitative mRNA analysis by RNAscope in treated gastrocnemius.

FIG. 9 shows a bar graph of protein quantification in tissues most affected in Danon disease by ELISA.

FIG. 10A shows a bar graph of protein quantification in regions of the heart by ELISA.

FIG. 10B shows a bar graph of protein quantification in muscles by ELISA.

FIGS. 11A-11D show line graphs of clinical pathology measurement in NHP serum over course of study. Clinical pathology levels were assessed as changes in (FIG. 11A) alanine aminotransferase, ALT; (FIG. 11B) aspartate aminotransferase, AST; (FIG. 11C) white blood cells, WBC; and (FIG. 11D) neutrophils over the study duration.

### DETAILED DESCRIPTION OF THE INVENTION

The present disclosure provides AAVrh74-based gene therapy vectors that employ optimized expression cassettes to deliver a polynucleotide encoding one of the Lysosome-associated membrane protein 2 (LAMP2) proteins, also known as CD107b. Generally, the LAMP2 is a human LAMP2, though expression of any mammalian LAMP-2 is envisioned. The native LAMP2 gene encodes by alternative splicing three variants: LAMP-2A, LAMP-2B, and LAMP-2C. LAMP-2B is associated with Danon disease. Although the disclosure concerns primarily Danon disease, LAMP2 is implicated in various other disease, including cancer. The disclosed vectors may be used to treat any of these diseases.

The disclosure further relates to AAVrh74 capsids or capsids having substantial homology to the AAVrh74 capsid and retaining the function of the AAVrh74 capsid. The disclosure provides the sequences listed in Table 1. Table 1 further provides polynucleotide sequences used in various embodiments. The sequences are not intended to limit the invention, as substitution or modification of these sequences with different promoters, enhancer, or other genetic elements is contemplated.

TABLE 1
Sequences

| SEQ ID NO: | Type | Description |
|---|---|---|
| 1 | nucleotide | AAVrh74 capsid coding sequence |
| 2 | protein | AAVrh74 VP1 |
| 3 | protein | AAVrh74 VP2 |
| 4 | protein | AAVrh74 VP3 |
| 5 | protein | LAMP-2B (wild-type) |
| 6 | nucleotide | LAMP-2B coding sequence (wild-type) |
| 7 | nucleotide | LAMP-2B engineered coding sequence |
| 8 | nucleotide | LAMP-2B engineered coding sequence |
| 9 | nucleotide | LAMP-2B engineered coding sequence |
| 10 | nucleotide | Engineered expression cassette |
| 11 | nucleotide | Engineered expression cassette |
| 12 | nucleotide | Engineered expression cassette |
| 13 | nucleotide | AAV inverted terminal repeat |
| 14 | nucleotide | AAV inverted terminal repeat |
| 15 | protein | LAMP-2A protein sequence |
| 16 | protein | LAMP-2B protein sequence |
| 17 | protein | LAMP-2C protein sequence |
| 18 | nucleotide | CAG promoter |
| 19 | nucleotide | WPRE |
| 20 | nucleotide | Kozak sequence |
| 21 | nucleotide | Kozak sequence |
| 22 | nucleotide | Kozak sequence |
| 23 | nucleotide | Kozak sequence |
| 24 | nucleotide | Kozak sequence |
| 25 | nucleotide | Kozak sequence |
| 26 | nucleotide | polyadenylation signal (full length) |
| 27 | nucleotide | bGH polyadenylation signal (bGHpA) |
| 28 | nucleotide | SV40 early/late polyadenylation signal |
| 29 | nucleotide | human growth hormone (HGH) |
| | | polyadenylation signal |

The disclosure provides recombinant adeno-associated virus (rAAV) gene therapy vectors. As used herein, an “rAAV gene therapy vector” refers to a complete virus including nucleic acid and protein components, including capsid proteins. In some embodiments, the capsid protein is encoded by a polynucleotide supplied on a plasmid in trans to the transfer plasmid. The polynucleotide sequence of wild-type AAVrh74 cap is as follows:

AAVrh74 capsid coding sequence
(SEQ ID NO: 1)
ATGGCTGCCGATGGTTATCTTCCAGATTGGCTCGAGGACAACCTCTCTGA
GGGCATTCGCGAGTGGTGGGACCTGAAACCTGGAGCCCCGAAACCCAAAG
CCAACCAGCAAAAGCAGGACAACGGCCGGGGTCTGGTGCTTCCTGGCTAC
AAGTACCTCGGACCCTTCAACGGACTCGACAAGGGGGAGCCCGTCAACGC
GGCGGACGCAGCGGCCCTCGAGCACGACAAGGCCTACGACCAGCAGCTCC
AAGCGGGTGACAATCCGTACCTGCGGTATAATCACGCCGACGCCGAGTTT
CAGGAGCGTCTGCAAGAAGATACGTCTTTTGGGGGCAACCTCGGGCGCGC
AGTCTTCCAGGCCAAAAAGCGGGTTCTCGAACCTCTGGGCCTGGTTGAAT
CGCCGGTTAAGACGGCTCCTGGAAAGAAGAGACCGGTAGAGCCATCACCC
CAGCGCTCTCCAGACTCCTCTACGGGCATCGGCAAGAAAGGCCAGCAGCC
CGCAAAAAAGAGACTCAATTTTGGGCAGACTGGCGACTCAGAGTCAGTCC
CCGACCCTCAACCAATCGGAGAACCACCAGCAGGCCCCTCTGGTCTGGGA
TCTGGTACAATGGCTGCAGGCGGTGGCGCTCCAATGGCAGACAATAACGA
AGGCGCCGACGGAGTGGGTAGTTCCTCAGGAAATTGGCATTGCGATTCCA
CATGGCTGGGCGACAGAGTCATCACCACCAGCACCCGCACCTGGGCCCTG
CCCACCTACAACAACCACCTCTACAAGCAAATCTCCAACGGGACCTCGGG
AGGAAGCACCAACGACAACACCTACTTCGGCTACAGCACCCCCTGGGGGT
ATTTTGACTTCAACAGATTCCACTGCCACTTTTCACCACGTGACTGGCAG
CGACTCATCAACAACAACTGGGGATTCCGGCCCAAGAGGCTCAACTTCAA
GCTCTTCAACATCCAAGTCAAGGAGGTCACGCAGAATGAAGGCACCAAGA
CCATCGCCAATAACCTTACCAGCACGATTCAGGTCTTTACGGACTCGGAA
TACCAGCTCCCGTACGTGCTCGGCTCGGCGCACCAGGGCTGCCTGCCTCC
GTTCCCGGCGGACGTCTTCATGATTCCTCAGTACGGGTACCTGACTCTGA
ACAATGGCAGTCAGGCTGTGGGCCGGTCGTCCTTCTACTGCCTGGAGTAC
TTTCCTTCTCAAATGCTGAGAACGGGCAACAACTTTGAATTCAGCTACAA
CTTCGAGGACGTGCCCTTCCACAGCAGCTACGCGCACAGCCAGAGCCTGG
ACCGGCTGATGAACCCTCTCATCGACCAGTACTTGTACTACCTGTCCCGG
ACTCAAAGCACGGGCGGTACTGCAGGAACTCAGCAGTTGCTATTTTCTCA
GGCCGGGCCTAACAACATGTCGGCTCAGGCCAAGAACTGGCTACCCGGTC
CCTGCTACCGGCAGCAACGCGTCTCCACGACACTGTCGCAGAACAACAAC
AGCAACTTTGCCTGGACGGGTGCCACCAAGTATCATCTGAATGGCAGAGA
CTCTCTGGTGAATCCTGGCGTTGCCATGGCTACCCACAAGGACGACGAAG
AGCGATTTTTTCCATCCAGCGGAGTCTTAATGTTTGGGAAACAGGGAGCT
GGAAAAGACAACGTGGACTATAGCAGCGTGATGCTAACCAGCGAGGAAGA
AATAAAGACCACCAACCCAGTGGCCACAGAACAGTACGGCGTGGTGGCCG
ATAACCTGCAACAGCAAAACGCCGCTCCTATTGTAGGGGCCGTCAATAGT
CAAGGAGCCTTACCTGGCATGGTGTGGCAGAACCGGGACGTGTACCTGCA
GGGTCCCATCTGGGCCAAGATTCCTCATACGGACGGCAACTTTCATCCCT
CGCCGCTGATGGGAGGCTTTGGACTGAAGCATCCGCCTCCTCAGATCCTG
ATTAAAAACACACCTGTTCCCGCGGATCCTCCGACCACCTTCAATCAGGC
CAAGCTGGCTTCTTTCATCACGCAGTACAGTACCGGCCAGGTCAGCGTGG
AGATCGAGTGGGAGCTGCAGAAGGAGAACAGCAAACGCTGGAACCCAGAG
ATTCAGTACACTTCCAACTACTACAAATCTACAAATGTGGACTTTGCTGT
CAATACTGAGGGTACTTATTCCGAGCCTCGCCCCATTGGCACCCGTTACC
TCACCCGTAATCTGTAA

The disclosure further provides protein sequences for AAVrh74 VP1, VP2, and VP3, including SEQ ID NOs: 2-4, and homologs or functional variants thereof.

AAVrh74 VP1
(SEQ ID NO: 2)
MAAGGGAPMADNNEGADGVGSSSGNWHCDSTWLGDRVITTSTRTWALPTY
NNHLYKQISNGTSGGSTNDNTYFGYSTPWGYFDFNRFHCHFSPRDWQRLI
NNNWGFRPKRLNFKLFNIQVKEVTQNEGTKTIANNLTSTIQVFTDSEYQL
PYVLGSAHQGCLPPFPADVFMIPQYGYLTLNNGSQAVGRSSFYCLEYFPS
QMLRTGNNFEFSYNFEDVPFHSSYAHSQSLDRLMNPLIDQYLYYLSRTQS
TGGTAGTQQLLFSQAGPNNMSAQAKNWLPGPCYRQQRVSTTLSQNNNSNF
AWTGATKYHLNGRDSLVNPGVAMATHKDDEERFFPSSGVLMFGKQGAGKD
NVDYSSVMLTSEEEIKTTNPVATEQYGVVADNLQQQNAAPIVGAVNSQGA
LPGMVWQNRDVYLQGPIWAKIPHTDGNFHPSPLMGGFGLKHPPPQILIKN
TPVPADPPTTFNQAKLASFITQYSTGQVSVEIEWELQKENSKRWNPEIQY
TSNYYKSTNVDFAVNTEGTYSEPRPIGTRYLTRNL
AAVrh74 VP2
(SEQ ID NO: 3)
STIQVFTDSEYQLPYVLGSAHQGCLPPFPADVFMIPQYGYLTLNNGSQAV
GRSSFYCLEYFPSQMLRTGNNFEFSYNFEDVPFHSSYAHSQSLDRLMNPL
IDQYLYYLSRTQSTGGTAGTQQLLFSQAGPNNMSAQAKNWLPGPCYRQQR
VSTTLSQNNNSNFAWTGATKYHLNGRDSLVNPGVAMATHKDDEERFFPSS
GVLMFGKQGAGKDNVDYSSVMLTSEEEIKTTNPVATEQYGVVADNLQQQN
AAPIVGAVNSQGALPGMVWQNRDVYLQGPIWAKIPHTDGNFHPSPLMGGF
GLKHPPPQILIKNTPVPADPPTTFNQAKLASFITQYSTGQVSVEIEWELQ
KENSKRWNPEIQYTSNYYKSTNVDFAVNTEGTYSEPRPIGTRYLTRNL
AAVrh74 VP3
(SEQ ID NO: 4)
RTGNNFEFSYNFEDVPFHSSYAHSQSLDRLMNPLIDQYLYYLSRTQSTGG
TAGTQQLLFSQAGPNNMSAQAKNWLPGPCYRQQRVSTTLSQNNNSNFAWT
GATKYHLNGRDSLVNPGVAMATHKDDEERFFPSSGVLMFGKQGAGKDNVD
YSSVMLTSEEEIKTTNPVATEQYGVVADNLQQQNAAPIVGAVNSQGALPG
MVWQNRDVYLQGPIWAKIPHTDGNFHPSPLMGGFGLKHPPPQILIKNTPV
PADPPTTFNQAKLASFITQYSTGQVSVEIEWELQKENSKRWNPEIQYTSN
YYKSTNVDFAVNTEGTYSEPRPIGTRYLTRNL

In certain cases, the AAVrh74 capsid comprises the amino acid sequence set forth in SEQ ID NO: 2. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh74 VP1 which is set forth in SEQ ID NO: 2. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh74 VP2 which is set forth in SEQ ID NO: 3. In some embodiments, the rAAV vector comprises a polypeptide that comprises, or consists essentially of, or yet further consists of a sequence, e.g., at least 65%, at least 70%, at least 75%, at least 80%, 81%, 82%, 83%, 84%, 85%, 86%, 87%, 88%, or 89%, more typically 90%, 91%, 92%, 93%, 94%, 95%, 96%, 97%, 98%, 99% or more identical to amino acid sequence of AAVrh74 VP3 which is set forth in SEQ ID NO: 4.

The wild-type polypeptide sequence of LAMP-2B (SEQ ID NO: 5) and the wild-type polynucleotide sequence of LAMP-2B (SEQ ID NO: 6) are, respectively:

(SEQ ID NO: 5)
MVCFRLFPVPGSGLVLVCLVLGAVRSYALELNLTDSENATCLYAKWQMNF
TVRYETTNKTYKTVTISDHGTVTYNGSICGDDQNGPKIAVQFGPGFSWIA
NFTKAASTYSIDSVSFSYNTGDNTTFPDAEDKGILTVDELLAIRIPLNDL
FRCNSLSTLEKNDVVQHYWDVLVQAFVQNGTVSTNEFLCDKDKTSTVAPT
IHTTVPSPTTTPTPKEKPEAGTYSVNNGNDTCLLATMGLQLNITQDKVAS
VININPNTTHSTGSCRSHTALLRLNSSTIKYLDFVFAVKNENRFYLKEVN
ISMYLVNGSVFSIANNNLSYWDAPLGSSYMCNKEQTVSVSGAFQINTFDL
RVQPFNVTQGKYSTAQECSLDDDTILIPIIVGAGLSGLIIVIVIAYVIGR
RKSYAGYQT;
and
(SEQ ID NO: 6)
ATGGTGTGCTTCCGCCTCTTCCCGGTTCCGGGCTCAGGGCTCGTTCTGGT
CTGCCTAGTCCTGGGAGCTGTGCGGTCTTATGCATTGGAACTTAATTTGA
CAGATTCAGAAAATGCCACTTGCCTTTATGCAAAATGGCAGATGAATTTC
ACAGTTCGCTATGAAACTACAAATAAAACTTATAAAACTGTAACCATTTC
AGACCATGGCACTGTGACATATAATGGAAGCATTTGTGGGGATGATCAGA
ATGGTCCCAAAATAGCAGTGCAGTTCGGACCTGGCTTTTCCTGGATTGCG
AATTTTACCAAGGCAGCATCTACTTATTCAATTGACAGCGTCTCATTTTC
CTACAACACTGGTGATAACACAACATTTCCTGATGCTGAAGATAAAGGAA
TTCTTACTGTTGATGAACTTTTGGCCATCAGAATTCCATTGAATGACCTT
TTTAGATGCAATAGTTTATCAACTTTGGAAAAGAATGATGTTGTCCAACA
CTACTGGGATGTTCTTGTACAAGCTTTTGTCCAAAATGGCACAGTGAGCA
CAAATGAGTTCCTGTGTGATAAAGACAAAACTTCAACAGTGGCACCCACC
ATACACACCACTGTGCCATCTCCTACTACAACACCTACTCCAAAGGAAAA
ACCAGAAGCTGGAACCTATTCAGTTAATAATGGCAATGATACTTGTCTGC
TGGCTACCATGGGGCTGCAGCTGAACATCACTCAGGATAAGGTTGCTTCA
GTTATTAACATCAACCCCAATACAACTCACTCCACAGGCAGCTGCCGTTC
TCACACTGCTCTACTTAGACTCAATAGCAGCACCATTAAGTATCTAGACT
TTGTCTTTGCTGTGAAAAATGAAAACCGATTTTATCTGAAGGAAGTGAAC
ATCAGCATGTATTTGGTTAATGGCTCCGTTTTCAGCATTGCAAATAACAA
TCTCAGCTACTGGGATGCCCCCCTGGGAAGTTCTTATATGTGCAACAAAG
AGCAGACTGTTTCAGTGTCTGGAGCATTTCAGATAAATACCTTTGATCTA
AGGGTTCAGCCTTTCAATGTGACACAAGGAAAGTATTCTACAGCCCAAGA
GTGTTCGCTGGATGATGACACCATTCTAATCCCAATTATAGTTGGTGCTG
GTCTTTCAGGCTTGATTATCGTTATAGTGATTGCTTACGTAATTGGCAGA
AGAAAAAGTTATGCTGGATATCAGACTCTGTAA.

In an embodiment, the transgene shares at least 95% identity to the polynucleotide sequence of SEQ ID NO: 5. In an embodiment, the transgene shares at least 99% identity to the polynucleotide sequence of SEQ ID NO: 5. In an embodiment, the transgene comprises the polynucleotide sequence of SEQ ID NO: 5. In embodiment, the transgene shares at least 85%, 86%, 87%, 88%, 89%, 90%, 91%, 92%, 93%, 94%, 95%, 96%, 97%, 98%, 99%, or complete identity to SEQ ID NO:5.

In an embodiment, the transgene encodes a polypeptide that shares at least 95% identity to the amino acid sequence of SEQ ID NO: 6. In an embodiment, the transgene encodes a polypeptide shares at least 99% identity to the amino acid sequence of SEQ ID NO: 6. In an embodiment, the polypeptide encoded by the transgene comprises the amino acid sequence of SEQ ID NO: 6. In embodiment, the polypeptide encoded by the transgene shares at least 85%, 86%, 87%, 88%, 89%, 90%, 91%, 92%, 93%, 94%, 95%, 96%, 97%, 98%, 99%, or complete identity to SEQ ID NO:6.

Disclosed herein are modifications to the gene sequence of LAMP-2B including: codon-optimization, CpG depletion, removal of cryptic splice sites, and reduction of alternative open-reading frames (ORFs). In embodiments, the disclosure provides a transgene encoding an isoform of lysosome-associated membrane protein 2 (LAMP-2) or a functional variant thereof. In embodiments, the transgene shares at least 85%, 86%, 87%, 88%, 89%, 90%, 91%, 92%, 93%, 94%, 95%, 96%, 97%, 98%, 99%, or complete identity to a sequence selected from SEQ ID NO: 7-9. The disclosure provides at least three variant gene sequences for LAMP-2B (SEQ ID NO: 7-9):

(SEQ ID NO: 7)
ATGGTCTGCTTCAGACTGTTCCCTGTCCCTGGATCTGGTCTGGTGCTTGT
GTGCTTGGTGCTGGGTGCTGTGAGATCCTATGCCCTTGAGCTGAACCTGA
CTGACTCAGAAAATGCCACTTGCCTGTATGCCAAGTGGCAGATGAACTTC
ACTGTGAGATATGAGACTACCAACAAGACCTACAAGACTGTGACCATCTC
AGACCATGGCACTGTCACCTACAATGGATCAATCTGTGGTGATGATCAGA
ATGGCCCAAAGATAGCAGTGCAGTTTGGGCCCGGTTTTTCCTGGATTGCT
AACTTCACCAAGGCAGCCTCCACCTACAGCATTGACTCAGTCAGCTTCAG
CTACAACACTGGGGATAACACCACCTTCCCTGACGCAGAGGACAAGGGAA
TCCTTACTGTGGACGAACTCCTGGCAATCAGAATCCCCCTTAACGACCTG
TTCAGATGCAACTCCCTTTCAACCCTTGAAAAGAATGATGTGGTGCAACA
CTATTGGGACGTCCTGGTGCAAGCCTTTGTGCAGAATGGGACAGTGAGTA
CCAACGAGTTCCTCTGTGACAAGGACAAGACCAGCACTGTGGCCCCCACT
ATCCACACCACTGTGCCCAGCCCTACCACTACCCCCACCCCTAAAGAGAA
GCCAGAAGCTGGAACCTACTCAGTCAACAATGGAAATGACACATGCCTCC
TTGCCACCATGGGACTGCAGCTGAACATCACTCAGGACAAGGTGGCCTCA
GTGATTAACATCAACCCTAACACCACTCATAGCACTGGGAGCTGCAGATC
ACATACAGCTCTGCTGAGGCTCAACTCCTCCACCATCAAGTACCTGGACT
TTGTGTTTGCTGTGAAGAATGAGAACAGGTTCTACCTCAAGGAAGTGAAC
ATTTCCATGTACCTGGTCAATGGTTCAGTGTTCTCTATTGCCAACAACAA
TCTGAGCTACTGGGATGCACCCCTGGGATCCTCCTACATGTGCAACAAGG
AGCAGACTGTGAGTGTGTCAGGTGCTTTTCAGATCAACACTTTTGACCTG
AGGGTGCAGCCCTTCAATGTGACTCAGGGAAAGTACTCCACTGCACAAGA
GTGTTCCTTGGATGATGACACTATCCTCATCCCCATTATTGTGGGAGCTG
GACTGTCAGGATTGATTATAGTGATTGTGATTGCTTATGTGATTGGAAGG
AGAAAGAGCTATGCTGGCTACCAGACCCTGTAA;
(SEQ ID NO: 8)
ATGGTGTGCTTTAGACTGTTTCCTGTGCCTGGTTCAGGGCTGGTCCTGGT
CTGTCTGGTGCTGGGGGCTGTCAGAAGCTATGCCTTGGAGCTGAACCTCA
CTGATAGTGAAAATGCCACTTGTCTGTATGCTAAGTGGCAGATGAACTTC
ACTGTGAGATATGAAACCACCAACAAGACTTACAAAACAGTGACCATCTC
AGATCATGGAACTGTGACCTACAACGGCAGCATTTGTGGAGACGACCAGA
ACGGACCAAAAATCGCTGTCCAATTTGGGCCTGGATTCTCCTGGATTGCC
AATTTCACTAAAGCTGCCTCCACATATTCAATTGACTCAGTGTCCTTCTC
CTACAACACTGGGGACAACACTACTTTCCCTGATGCTGAAGATAAGGGAA
TCTTGACAGTGGATGAGCTGCTGGCTATCAGGATCCCTTTGAATGACCTG
TTTAGGTGTAATTCACTGAGCACTCTGGAGAAGAACGACGTGGTGCAGCA
CTACTGGGACGTGCTGGTGCAGGCCTTTGTGCAGAACGGCACTGTGTCCA
CCAACGAATTCCTGTGTGATAAGGACAAAACTTCCACTGTGGCACCTACA
ATTCACACTACTGTGCCTTCACCTACCACCACTCCAACTCCAAAGGAAAA
GCCTGAAGCAGGAACCTACTCTGTGAACAATGGCAATGATACCTGTCTGT
TGGCCACCATGGGCCTCCAACTGAACATTACTCAGGACAAGGTGGCCTCA
GTGATTAACATTAACCCCAACACTACCCACTCCACTGGCAGCTGTAGATC
ACACACAGCCTTGCTCAGACTGAATAGCAGCACCATCAAGTATTTGGATT
TTGTGTTTGCAGTGAAGAATGAAAACAGGTTCTACCTGAAGGAAGTCAAC
ATCTCAATGTACCTGGTGAACGGCTCAGTGTTCAGCATTGCCAACAACAA
CCTCTCCTATTGGGACGCTCCACTGGGGAGCAGCTACATGTGTAACAAGG
AACAGACTGTGTCAGTGTCAGGAGCCTTCCAGATTAACACCTTTGATCTG
AGGGTCCAACCCTTTAATGTCACTCAAGGAAAGTATAGCACTGCCCAGGA
GTGCTCCCTGGATGATGACACCATTCTGATTCCAATCATTGTGGGTGCAG
GACTTTCTGGGCTTATTATTGTGATTGTGATTGCCTATGTGATTGGCAGA
AGGAAATCCTATGCAGGGTACCAAACTCTGTAA;
or
(SEQ ID NO: 9)
ATGGTCTGTTTTAGGCTGTTCCCTGTCCCTGGTTCAGGACTGGTCTTAGT
GTGTCTGGTGCTTGGAGCTGTCAGAAGCTATGCCCTGGAGCTGAACCTGA
CTGACTCAGAAAATGCCACTTGCCTGTATGCCAAGTGGCAGATGAACTTC
ACTGTCAGATATGAAACCACCAACAAGACCTATAAGACTGTGACCATCTC
AGACCATGGCACTGTGACTTACAATGGGTCAATTTGTGGAGATGACCAGA
ATGGCCCTAAGATAGCTGTCCAGTTTGGTCCAGGATTCAGCTGGATTGCC
AACTTCACCAAGGCAGCCAGCACCTACAGCATTGACTCTGTGTCCTTCTC
CTACAACACAGGAGACAACACCACTTTCCCTGATGCAGAGGACAAAGGTA
TCCTGACTGTGGATGAGTTGCTGGCAATCAGGATCCCACTGAACGATCTG
TTCAGGTGCAACTCACTGTCCACTCTGGAAAAGAATGATGTGGTGCAGCA
CTATTGGGATGTGCTAGTCCAGGCCTTTGTCCAGAATGGGACTGTGTCAA
CTAATGAGTTCCTGTGTGACAAGGACAAGACAAGCACTGTAGCCCCCACT
ATCCATACCACAGTACCTAGCCCCACCACTACTCCAACCCCCAAGGAGAA
GCCTGAGGCTGGCACCTACTCAGTGAACAATGGGAATGACACCTGTTTGC
TGGCCACTATGGGACTCCAACTGAACATCACCCAGGACAAAGTGGCCTCT
GTGATCAATATCAATCCCAACACCACCCACAGCACTGGGTCCTGCAGAAG
CCACACTGCCCTCCTGAGGCTCAACTCATCAACTATCAAGTACTTGGATT
TTGTGTTTGCAGTGAAGAATGAGAACAGATTCTACCTCAAAGAGGTCAAC
ATTTCAATGTACCTGGTGAATGGGAGTGTGTTCTCCATTGCTAACAACAA
CCTGAGCTACTGGGATGCCCCTCTGGGCTCCTCATACATGTGCAACAAGG
AACAGACTGTGAGTGTGTCAGGGGCCTTCCAGATCAACACTTTTGACCTG
AGAGTGCAGCCCTTTAATGTGACACAGGGAAAGTACAGCACTGCTCAGGA
GTGCAGCCTGGATGATGACACTATCCTGATCCCTATCATTGTGGGGGCAG
GCCTGTCTGGACTCATTATTGTGATTGTGATTGCCTATGTGATAGGGAGA
AGGAAGTCTTATGCTGGATACCAGACCCTGTAA.

In an embodiment, the transgene shares at least 95% identity to a sequence selected from SEQ ID NO: 7-9. In an embodiment, the transgene shares at least 99% identity to a sequence selected from SEQ ID NO: 7-9. In an embodiment, the transgene comprises a sequence selected from SEQ ID NO: 7-9. In embodiment, the transgene shares at least 85%, 86%, 87%, 88%, 89%, 90%, 91%, 92%, 93%, 94%, 95%, 96%, 97%, 98%, 99%, or complete identity to SEQ ID NO: 7. In embodiment, the transgene shares at least 85%, 86%, 87%, 88%, 89%, 90%, 91%, 92%, 93%, 94%, 95%, 96%, 97%, 98%, 99%, or complete identity to SEQ ID NO: 8. In embodiment, the transgene shares at least 85%, 86%, 87%, 88%, 89%, 90%, 91%, 92%, 93%, 94%, 95%, 96%, 97%, 98%, 99%, or complete identity to SEQ ID NO: 9.

In some cases, the transgene has a polynucleotide sequence that is different from the polynucleotide sequence of a reference sequence, e.g., a “native” or “wild-type” LAMP-2B sequence. In some embodiments, the transgene shares at most 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, or 95% identity with a reference sequence. In some embodiments, the reference sequence is SEQ ID NO: 6. For example, SEQ ID NO: 7 shares 78.5% identity to SEQ ID NO: 6.

In some embodiments, the transgene is similar to or identical to a subsequence of any one of SEQ ID NOs: 5 or 7-9. In some embodiments, the transgene comprises a subsequence of any one of SEQ ID NOs: 5 or 7-9. In various embodiments, the subsequence may comprise any set of consecutive nucleotides (nt) in the full sequence having a length of at least about 50 nt, at least about 100 nt, at least about 150 nt, at least about 250 nt, at least about 200 nt, at least about 350 nt, at least about 450 nt, at least about 400 nt, at least about 450 nt, at least about 550 nt, at least about 600 nt, at least about 650 nt, at least about 600 nt, at least about 650 nt, at least about 700 nt, at least about 750 nt, at least about 800 nt, at least about 850 nt, at least about 900 nt, at least about 950 nt, at least about 1000 nt, at least about 1050 nt, at least about 1100 nt, at least about 1150 nt, or at least about 1200 nt.

In some embodiments, the transgene encodes a polypeptide similar to or identical to a subsequence of any one of SEQ ID NOs: 6 or 16-18. In some embodiments, the transgene encodes a polypeptide comprises a subsequence of any one of SEQ ID NOs: 6 or 16-18. In some embodiments, the subsequence may comprises any set of consecutive amino acids (aa) in the full sequence having a length of at least about 20 aa, at least about 30 aa, at least about 50 aa, at least about 70 aa, at least about 80 aa, at least about 100 aa, at least about 120 aa, at least about 130 aa, at least about 150 aa, at least about 170 aa, at least about 180 aa, at least about 200 aa, at least about 220 aa, at least about 230 aa, at least about 250 aa, at least about 270 aa, at least about 280 aa, at least about 300 aa, at least about 320 aa, at least about 330 aa, at least about 350 aa, at least about 370 aa, at least about 380 aa, or at least about 400 aa.

In some embodiments, the transgene encodes a LAMP-2 polypeptide comprising an N-terminal truncation 1 to 10 amino acids (aa), 1 to 20 aa, 1 to 30 aa, 1 to 40 aa, or 1 to 50 aa, or an N-terminal truncation 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 48, 50, or more aa; and/or a C-terminal truncation 1 to 10 amino acids (aa), 1 to 20 aa, 1 to 30 aa, 1 to 40 aa, or 1 to 50 aa, or a C-terminal truncation 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 48, 50, or more aa.

In some embodiments, the subsequence of the LAMP2 polypeptide comprises a functional variant of LAMP-2A, LAMP-2B, or LAMP-2C. As used herein, a “functional variant” refers to polypeptide sharing sequence similarity to a reference LAMP-2A, LAMP-2B, or LAMP-2C and having at least one biological property of LAMP-2A, LAMP-2B, or LAMP-2C. The biological property may include the ability to specifically interact with one or more binding partners, the ability to bind an anti-LAMP2 antibody, and/or the ability to complement a defect in LAMP2 activity in a cell, tissue, and/or organism.

In some embodiments, the subsequence of the LAMP2 polypeptide comprises a functional fragment of LAMP-2A, LAMP-2B, or LAMP-2C. As used herein, a “functional fragment” refers to polypeptide sharing sequence similarity to a subsequence of a reference LAMP-2A, LAMP-2B, or LAMP-2C and having at least one biological property of LAMP-2A, LAMP-2B, or LAMP-2C. The biological property may include the ability to specifically interact with one or more binding partners, the ability to bind an anti-LAMP2 antibody, and/or the ability to complement a defect in LAMP2 activity in a cell, tissue, and/or organism.

In an embodiment, the transgene is codon-optimized for expression in a human host cell. In an embodiment, the transgene coding sequence is modified, or “codon optimized” to enhance expression by replacing infrequently represented codons with more frequently represented codons. The coding sequence is the portion of the mRNA sequence that encodes the amino acids for translation. During translation, each of 61 trinucleotide codons are translated to one of 20 amino acids, leading to a degeneracy, or redundancy, in the genetic code. However, different cell types, and different animal species, utilize tRNAs (each bearing an anticodon) coding for the same amino acids at different frequencies. When a gene sequence contains codons that are infrequently represented by the corresponding tRNA, the ribosome translation machinery may slow, impeding efficient translation. Expression can be improved via “codon optimization” for a particular species, where the coding sequence is altered to encode the same protein sequence, but utilizing codons that are highly represented, and/or utilized by highly expressed human proteins (Cid-Arregui et al., 2003; J. Virol. 77: 4928).

In some embodiments, the coding sequence of the transgene is modified to replace codons infrequently expressed in mammal or in primates with codons frequently expressed in primates. For example, in some embodiments, the transgene encodes a polypeptide having at least 85% sequence identity to a reference polypeptide (e.g. wild-type LAMP-2B; SEQ ID NO: 16)—for example, at least 90% sequence identity, at least 95% sequence identity, at least 98% identity, or at least 99% identity to the reference polypeptide—wherein at least one codon of the coding sequence has a higher tRNA frequency in humans than the corresponding codon in the sequence disclosed above or herein.

In an embodiment, the transgene comprises fewer alternative open reading frames than SEQ ID: 6. In an embodiment, the transgene is modified to enhance expression by termination or removal of open reading frames (ORFs) that do not encode the desired transgene. An open reading frame (ORF) is the nucleic acid sequence that follows a start codon and does not contain a stop codon. ORFs may be in the forward or reverse orientation, and may be “in frame” or “out of frame” compared with the gene of interest. Such open reading frames have the potential to be expressed in an expression cassette alongside the gene of interest, and could lead to undesired adverse effects. In some embodiments the transgene has been modified to remove open reading frames by further altering codon usage. This is done by eliminating one or more start codons (ATG, TTG, CTG) and/or introducing one or more stop codons (TAG, TAA, or TGA) in reverse orientation or out-of-frame to the desired ORF, while preserving the encoded amino acid sequence and, optionally, maintaining highly utilized codons in the gene of interest (i.e., avoiding codons with frequency <20%).

In variations of the present disclosure, the transgene coding sequence may be optimized by either of codon optimization and removal of non-transgene ORFs or using both techniques. In some cases, one removes or minimizes non-transgene ORFs after codon optimization in order to remove ORFs introduced during codon optimization.

In an embodiment, the transgene contains fewer CpG sites than SEQ ID: 6. Without being bound by theory, it is believed that the presence of CpG sites in a polynucleotide sequence is associated with the undesirable immunological responses of the host against a viral vector comprising the polynucleotide sequence. In some embodiments, the transgene is designed to reduce the number of CpG sites. Exemplary methods are provided in U.S. Patent Application Publication No. US20020065236A1.

In an embodiment, the transgene contains fewer cryptic splice sites than SEQ ID: 6. For the optimization, GeneArt® software may be used, e.g., to increase the GC content and/or remove cryptic splice sites in order to avoid transcriptional silencing and, therefore, increase transgene expression. Alternatively, any optimization method known in the art may be used. Removal of cryptic splice sites is described, for example, in International Patent Application Publication No. WO2004015106A1.

Also disclosed herein are expression cassettes and gene therapy vectors encoding LAMP-2B, e.g., a codon-optimized LAMP-2B sequence disclosed herein, comprising: a consensus optimal Kozak sequence, a full-length polyadenylation (polyA) sequence (or substitution of full-length polyA for a truncated polyA), and minimal or no upstream (i.e. 5′) start codons (i.e. ATG sites).

In some cases, the expression cassette contains two or more of a first inverted terminal repeat, an enhancer/promoter region, a consensus optimal Kozak sequence, a transgene (e.g., a transgene encoding a LAMP-2B disclosed herein), a 3′ untranslated region including a full-length polyA sequence, and a second inverted terminal repeat.

In an embodiment, the expression cassette comprises a Kozak sequence operatively linked to the transgene. In an embodiment, the Kozak sequence is a consensus optimal Kozak sequence comprising or consisting of SEQ ID NO: 20.

(SEQ ID NO: 20)
GCCGCCACCATGG

In various embodiments, the expression cassette comprises an alternative Kozak sequence operatively linked to the transgene. In an embodiment, the Kozak sequence is an alternative Kozak sequence comprising or consisting of any one of SEQ ID NOs. 21-25.

(SEQ ID NO: 21)
(gcc)gccRccAUGG
(SEQ ID NO: 22)
AGNNAUGN
(SEQ ID NO: 23)
ANNAUGG
(SEQ ID NO: 24)
ACCAUGG
(SEQ ID NO: 25)
GACACCAUGG

In SEQ ID NO: 21, a lower-case letter denotes the most common base at a position where the base can nevertheless vary; an upper-case letter indicates a highly conserved base; ‘R’ indicates adenine or guanine. In SEQ ID NO: 21, the sequence in parentheses (gcc) is optional. IN SEQ ID NOs: 22-23, ‘N’ denotes any base.

A variety of sequences can be used in place of this consensus optimal Kozak sequence as the translation-initiation site and it is within the skill of those in the art to identify and test other sequences. See Kozak M. An analysis of vertebrate mRNA sequences: intimations of translational control. J. Cell Biol. 115 (4): 887-903 (1991).

In an embodiment, the expression cassette comprises a full-length polyA sequence operatively linked to the transgene. In an embodiment, the full-length polyA sequence comprises SEQ ID NO: 26.

(SEQ ID NO: 26)
TGGCTAATAAAGGAAATTTATTTTCATTGCAATAGTGTGTTGGAATTTTT
TGTGTCTCTCACTCGGAAGGACATATGGGAGGGCAAATCATTTAAAACAT
CAGAATGAGTATTTGGTTTAGAGTTTGGCAACATATGCCCATATGCTGGC
TGCCATGAACAAAGGTTGGCTATAAAGAGGTCATCAGTATATGAAACAGC
CCCCTGCTGTCCATTCCTTATTCCATAGAAAAGCCTTGACTTGAGGTTAG
ATTTTTTTTATATTTTGTTTTGTGTTATTTTTTTCTTTAACATCCCTAAA
ATTTTCCTTACATGTTTTACTAGCCAGATTTTTCCTCCTCTCCTGACTAC
TCCCAGTCATAGCTGTCCCTCTTCTCTTATGGAGATC

Various alternative polyA sequences may be used in expression cassettes of the present disclosure, including without limitation, bovine growth hormone polyadenylation signal (bGHpA) (SEQ ID NO: 27), the SV40 early/late polyadenylation signal (SEQ ID NO: 28), and human growth hormone (HGH) polyadenylation signal (SEQ ID NO: 29).

(SEQ ID NO: 27)
TCGACTGTGCCTTCTAGTTGCCAGCCATCTGTTGTTTGCCCCTCCCCCGT
GCCTTCCTTGACCCTGGAAGGTGCCACTCCCACTGTCCTTTCCTAATAAA
ATGAGGAAATTGCATCGCATTGTCTGAGTAGGTGTCATTCTATTCTGGGG
GGTGGGGTGGGGCAGGACAGCAAGGGGGAGGATTGGGAGGACAATAGCAG
GCATGCTGGGGATGCGGTGGGCTCTATGGCTTCTG
(SEQ ID NO: 28)
CAGACATGATAAGATACATTGATGAGTTTGGACAAACCACAACTAGAATG
CAGTGAAAAAAATGCTTTATTTGTGAAATTTGTGATGCTATTGCTTTATT
TGTAACCATTATAAGCTGCAATAAACAAGTTAACAACAACAATTGCATTC
ATTTTATGTTTCAGGTTCAGGGGGAGATGTGGGAGGTTTTTTAAAGCAAG
TAAAACCTCTACAAATGTGGTA
(SEQ ID NO: 29)
CTGCCCGGGTGGCATCCCTGTGACCCCTCCCCAGTGCCTCTCCTGGCCCT
GGAAGTTGCCACTCCAGTGCCCACCAGCCTTGTCCTAATAAAATTAAGTT
GCATCATTTTGTCTGACTAGGTGTCCTTCTATAATATTATGGGGTGGAGG
GGGGTGGTATGGAGCAAGGGGCCCAAGTTGGGAAGAAACCTGTAGGGCCT
GC

In some embodiments, the expression cassette comprises an active fragment of a polyA sequence. In particular embodiments, the active fragment of the polyA sequence comprises or consists of less than 20 base pair (bp), less than 50 bp, less than 100 bp, or less than 150 bp, e.g., of any of the polyA sequences disclosed herein.

In some cases, expression of the transgene is increased by ensuring that the expression cassette does not contain competing ORFs. In an embodiment, the expression cassette comprises no start codon within 20, 30, 40, 50, 60, 70, 80, 90, 100, 200, or 300 base pairs 5′ of the start codon of the transgene. In an embodiment, the expression cassette comprises no start codon 5′ of the start codon of the transgene. In some embodiments, the expression cassette comprises no alternative transcripts. In some embodiments, the expression cassette comprises no alternative transcripts, except small transcripts, e.g. 300 base pairs or less.

In an embodiment, the expression cassette comprises operatively linked, in the 5′ to 3′ direction, a first inverted terminal repeat, an enhancer/promoter region, introns, a consensus optimal Kozak sequence, the transgene, a 3′ untranslated region including a full-length polyA sequence, and a second inverted terminal repeat, where the expression cassette comprises no start codon 5′ to the start codon of the transgene.

In an embodiment, the enhancer/promoter region comprises, in the 5′ to 3′ direction: a CMV IE Enhancer and a Chicken Beta-Actin Promoter. In an embodiment, the enhancer/promoter region comprises a CAG promoter. As used herein “CAG promoter” refers to a polynucleotide sequence comprising a CMV early enhancer element, a chicken beta-actin promoter, the first exon and first intron of the chicken beta-actin gene, and a splice acceptor from the rabbit beta-globin gene.

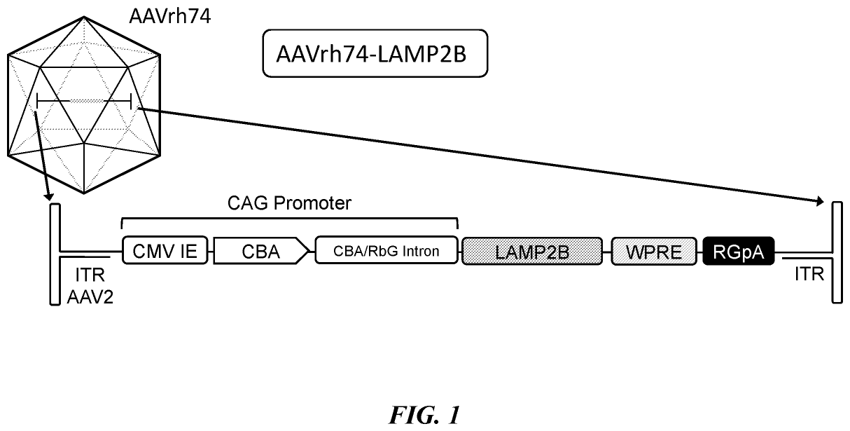

In an embodiment, the expression cassette shares at least 95% identity to a sequence selected from SEQ ID NOs: 10-12. In an embodiment, the expression cassette shares complete identity to a sequence selected from SEQ ID NOs: 10-12, or shares at least 80%, at least 85%, at least 90%, at least 95%, at least 98%, or at least 99% identity to a sequence selected from SEQ ID NOs: 10-12. In certain embodiments, the expression cassette comprises one or more modifications as compared to a sequence selected from SEQ ID NOs: 10-12. In particular embodiments, the one or more modifications comprises one or more of: removal of one or more (e.g., all) upstream ATG sequences, replacement of the Kozak sequence with an optimized consensus Kozak sequence or another Kozak sequence, including but not limited to any of those disclosed herein, and/or replacement of the polyadenylation sequence with a full-length polyadenylation sequence or another polyadenylation sequence, including but not limited to any of those disclosed herein. An illustrative configuration of genetic elements within these exemplary expression cassettes is depicted in FIG. 1.

(SEQ ID NO: 10)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTTGTAGTTAATGATTAACCCG
CCATGCTACTTATCTACCAGGGTAATGGGGATCCTCTAGAACTATAGCTA
GTCGACATTGATTATTGACTAGTTATTAATAGTAATCAATTACGGGGTCA
TTAGTTCATAGCCCATATATGGAGTTCCGCGTTACATAACTTACGGTAAA
TGGCCCGCCTGGCTGACCGCCCAACGACCCCCGCCCATTGACGTCAATAA
TGACGTATGTTCCCATAGTAACGCCAATAGGGACTTTCCATTGACGTCAA
TGGGTGGAGTATTTACGGTAAACTGCCCACTTGGCAGTACATCAAGTGTA
TCATATGCCAAGTACGCCCCCTATTGACGTCAATGACGGTAAATGGCCCG
CCTGGCATTATGCCCAGTACATGACCTTATGGGACTTTCCTACTTGGCAG
TACATCTACGTATTAGTCATCGCTATTACCATGGTCGAGGTGAGCCCCAC
GTTCTGCTTCACTCTCCCCATCTCCCCCCCCTCCCCACCCCCAATTTTGT
ATTTATTTATTTTTTAATTATTTTGTGCAGCGATGGGGGCGGGGGGGGGG
GGGGGGCGCGCGCCAGGCGGGGCGGGGCGGGGCGAGGGGCGGGGCGGGGC
GAGGCGGAGAGGTGCGGCGGCAGCCAATCAGAGCGGCGCGCTCCGAAAGT
TTCCTTTTATGGCGAGGCGGCGGCGGCGGCGGCCCTATAAAAAGCGAAGC
GCGCGGCGGGCGGGAGTCGCTGCGCGCTGCCTTCGCCCCGTGCCCCGCTC
CGCCGCCGCCTCGCGCCGCCCGCCCCGGCTCTGACTGACCGCGTTACTCC
CACAGGTGAGCGGGCGGGACGGCCCTTCTCCTCCGGGCTGTAATTAGCGC
TTGGTTTAATGACGGCTTGTTTCTTTTCTGTGGCTGCGTGAAAGCCTTGA
GGGGCTCCGGGAGGGCCCTTTGTGCGGGGGGAGCGGCTCGGGGGGTGCGT
GCGTGTGTGTGTGCGTGGGGAGCGCCGCGTGCGGCTCCGCGCTGCCCGGC
GGCTGTGAGCGCTGCGGGCGCGGCGCGGGGCTTTGTGCGCTCCGCAGTGT
GCGCGAGGGGAGCGCGGCCGGGGGCGGTGCCCCGCGGTGCGGGGGGGGCT
GCGAGGGGAACAAAGGCTGCGTGCGGGGTGTGTGCGTGGGGGGGTGAGCA
GGGGGTGTGGGCGCGTCGGTCGGGCTGCAACCCCCCCTGCACCCCCCTCC
CCGAGTTGCTGAGCACGGCCCGGCTTCGGGTGCGGGGCTCCGTACGGGGC
GTGGCGCGGGGCTCGCCGTGCCGGGCGGGGGGTGGCGGCAGGTGGGGGTG
CCGGGCGGGGCGGGGCCGCCTCGGGCCGGGGAGGGCTCGGGGGAGGGGCG
CGGCGGCCCCCGGAGCGCCGGCGGCTGTCGAGGCGCGGCGAGCCGCAGCC
ATTGCCTTTTATGGTAATCGTGCGAGAGGGCGCAGGGACTTCCTTTGTCC
CAAATCTGTGCGGAGCCGAAATCTGGGAGGCGCCGCCGCACCCCCTCTAG
CGGGCGCGGGGCGAAGCGGTGCGGCGCCGGCAGGAAGGAAATGGGCGGGG
AGGGCCTTCGTGCGTCGCCGCGCCGCCGTCCCCTTCTCCCTCTCCAGCCT
CGGGGCTGTCCGCGGGGGGACGGCTGCCTTCGGGGGGGACGGGGCAGGGC
GGGGTTCGGCTTCTGGCGTGTGACCGGCGGCTCTAGAGCCTCTGCTAACC
ATGTTCATGCCTTCTTCTTTTTCCTACAGCTCCTGGGCAACGTGCTGGTT
ATTGTGCTGTCTCATCATTTTGGCAAAGAATTCGAGCGGCCGCCAGCCGC
CACCATGGTCTGCTTCAGACTGTTCCCTGTCCCTGGATCTGGTCTGGTGC
TTGTGTGCTTGGTGCTGGGTGCTGTGAGATCCTATGCCCTTGAGCTGAAC
CTGACTGACTCAGAAAATGCCACTTGCCTGTATGCCAAGTGGCAGATGAA
CTTCACTGTGAGATATGAGACTACCAACAAGACCTACAAGACTGTGACCA
TCTCAGACCATGGCACTGTCACCTACAATGGATCAATCTGTGGTGATGAT
CAGAATGGCCCAAAGATAGCAGTGCAGTTTGGGCCCGGTTTTTCCTGGAT
TGCTAACTTCACCAAGGCAGCCTCCACCTACAGCATTGACTCAGTCAGCT
TCAGCTACAACACTGGGGATAACACCACCTTCCCTGACGCAGAGGACAAG
GGAATCCTTACTGTGGACGAACTCCTGGCAATCAGAATCCCCCTTAACGA
CCTGTTCAGATGCAACTCCCTTTCAACCCTTGAAAAGAATGATGTGGTGC
AACACTATTGGGACGTCCTGGTGCAAGCCTTTGTGCAGAATGGGACAGTG
AGTACCAACGAGTTCCTCTGTGACAAGGACAAGACCAGCACTGTGGCCCC
CACTATCCACACCACTGTGCCCAGCCCTACCACTACCCCCACCCCTAAAG
AGAAGCCAGAAGCTGGAACCTACTCAGTCAACAATGGAAATGACACATGC
CTCCTTGCCACCATGGGACTGCAGCTGAACATCACTCAGGACAAGGTGGC
CTCAGTGATTAACATCAACCCTAACACCACTCATAGCACTGGGAGCTGCA
GATCACATACAGCTCTGCTGAGGCTCAACTCCTCCACCATCAAGTACCTG
GACTTTGTGTTTGCTGTGAAGAATGAGAACAGGTTCTACCTCAAGGAAGT
GAACATTTCCATGTACCTGGTCAATGGTTCAGTGTTCTCTATTGCCAACA
ACAATCTGAGCTACTGGGATGCACCCCTGGGATCCTCCTACATGTGCAAC
AAGGAGCAGACTGTGAGTGTGTCAGGTGCTTTTCAGATCAACACTTTTGA
CCTGAGGGTGCAGCCCTTCAATGTGACTCAGGGAAAGTACTCCACTGCAC
AAGAGTGTTCCTTGGATGATGACACTATCCTCATCCCCATTATTGTGGGA
GCTGGACTGTCAGGATTGATTATAGTGATTGTGATTGCTTATGTGATTGG
AAGGAGAAAGAGCTATGCTGGCTACCAGACCCTGTAAAAGGGCGAATTCC
AGCACACGCGTCCTAGGAGCTCGAGTACTACTGGCGGCCGTTACTAGTGG
ATCCGCGGTACAAGTAAGCATGCAAGCTTCGAGGACGGGGTGAACTACGC
CTGAATCAAGCTTATCGATAAATTCGAGCATCTTACCGCCATTTATTCCC
ATATTTGTTCTGTTTTTCTTGATTTGGGTATACATTTAAATGTTAATAAA
ACAAAATGGTGGGGCAATCATTTACATTTTTAGGGATATGTAATTACTAG
TTCAGGTGTATTGCCACAAGACAAACATGTTAAGAAACTTTCCCGTTATT
TACGCTCTGTTCCTGTTAATCAACCTCTGGATTACAAAATTTGTGAAAGA
TTGACTGATATTCTTAACTATGTTGCTCCTTTTACGCTGTGTGGATATGC
TGCTTTAATGCCTCTGTATCATGCTATTGCTTCCCGTACGGCTTTCGTTT
TCTCCTCCTTGTATAAATCCTGGTTGCTGTCTCTTTATGAGGAGTTGTGG
CCCGTTGTCCGTCAACGTGGCGTGGTGTGCTCTGTGTTTGCTGACGCAAC
CCCCACTGGCTGGGGCATTGCCACCACCTGTCAACTCCTTTCTGGGACTT
TCGCTTTCCCCCTCCCGATCGCCACGGCAGAACTCATCGCCGCCTGCCTT
GCCCGCTGCTGGACAGGGGCTAGGTTGCTGGGCACTGATAATTCCGTGGT
GTTGTCGGGGAAGGGCCTCGATACCGTCGATATCGATCCTGGCTAATAAA
GGAAATTTATTTTCATTGCAATAGTGTGTTGGAATTTTTTGTGTCTCTCA
CTCGGAAGGACATATGGGAGGGCAAATCATTTAAAACATCAGAATGAGTA
TTTGGTTTAGAGTTTGGCAACATATGCCCATATGCTGGCTGCCATGAACA
AAGGTTGGCTATAAAGAGGTCATCAGTATATGAAACAGCCCCCTGCTGTC
CATTCCTTATTCCATAGAAAAGCCTTGACTTGAGGTTAGATTTTTTTTAT
ATTTTGTTTTGTGTTATTTTTTTCTTTAACATCCCTAAAATTTTCCTTAC
ATGTTTTACTAGCCAGATTTTTCCTCCTCTCCTGACTACTCCCAGTCATA
GCTGTCCCTCTTCTCTTATGGAGATCGAAGCAATTCGTTGATCTGAATTT
CGACCACCCATAATAGATCTCCCATTACCCTGGTAGATAAGTAGCATGGC
GGGTTAATCATTAACTACAAGGAACCCCTAGTGATGGAGTTGGCCACTCC
CTCTCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGCAG
(SEQ ID NO: 11)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTTGTAGTTAATGATTAACCCG
CCATGCTACTTATCTACCAGGGTAATGGGGATCCTCTAGAACTATAGCTA
GTCGACATTGATTATTGACTAGTTATTAATAGTAATCAATTACGGGGTCA
TTAGTTCATAGCCCATATATGGAGTTCCGCGTTACATAACTTACGGTAAA
TGGCCCGCCTGGCTGACCGCCCAACGACCCCCGCCCATTGACGTCAATAA
TGACGTATGTTCCCATAGTAACGCCAATAGGGACTTTCCATTGACGTCAA
TGGGTGGAGTATTTACGGTAAACTGCCCACTTGGCAGTACATCAAGTGTA
TCATATGCCAAGTACGCCCCCTATTGACGTCAATGACGGTAAATGGCCCG
CCTGGCATTATGCCCAGTACATGACCTTATGGGACTTTCCTACTTGGCAG
TACATCTACGTATTAGTCATCGCTATTACCATGGTCGAGGTGAGCCCCAC
GTTCTGCTTCACTCTCCCCATCTCCCCCCCCTCCCCACCCCCAATTTTGT
ATTTATTTATTTTTTAATTATTTTGTGCAGCGATGGGGGCGGGGGGGGGG
GGGGGGCGCGCGCCAGGCGGGGCGGGGCGGGGCGAGGGGCGGGGCGGGGC
GAGGCGGAGAGGTGCGGCGGCAGCCAATCAGAGCGGCGCGCTCCGAAAGT
TTCCTTTTATGGCGAGGCGGCGGCGGCGGCGGCCCTATAAAAAGCGAAGC
GCGCGGCGGGCGGGAGTCGCTGCGCGCTGCCTTCGCCCCGTGCCCCGCTC
CGCCGCCGCCTCGCGCCGCCCGCCCCGGCTCTGACTGACCGCGTTACTCC
CACAGGTGAGCGGGCGGGACGGCCCTTCTCCTCCGGGCTGTAATTAGCGC
TTGGTTTAATGACGGCTTGTTTCTTTTCTGTGGCTGCGTGAAAGCCTTGA
GGGGCTCCGGGAGGGCCCTTTGTGCGGGGGGAGCGGCTCGGGGGGTGCGT
GCGTGTGTGTGTGCGTGGGGAGCGCCGCGTGCGGCTCCGCGCTGCCCGGC
GGCTGTGAGCGCTGCGGGCGCGGCGCGGGGCTTTGTGCGCTCCGCAGTGT
GCGCGAGGGGAGCGCGGCCGGGGGCGGTGCCCCGCGGTGCGGGGGGGGCT
GCGAGGGGAACAAAGGCTGCGTGCGGGGTGTGTGCGTGGGGGGGTGAGCA
GGGGGTGTGGGCGCGTCGGTCGGGCTGCAACCCCCCCTGCACCCCCCTCC
CCGAGTTGCTGAGCACGGCCCGGCTTCGGGTGCGGGGCTCCGTACGGGGC
GTGGCGCGGGGCTCGCCGTGCCGGGCGGGGGGTGGCGGCAGGTGGGGGTG
CCGGGCGGGGCGGGGCCGCCTCGGGCCGGGGAGGGCTCGGGGGAGGGGCG
CGGCGGCCCCCGGAGCGCCGGCGGCTGTCGAGGCGCGGCGAGCCGCAGCC
ATTGCCTTTTATGGTAATCGTGCGAGAGGGCGCAGGGACTTCCTTTGTCC
CAAATCTGTGCGGAGCCGAAATCTGGGAGGCGCCGCCGCACCCCCTCTAG
CGGGCGCGGGGCGAAGCGGTGCGGCGCCGGCAGGAAGGAAATGGGCGGGG
AGGGCCTTCGTGCGTCGCCGCGCCGCCGTCCCCTTCTCCCTCTCCAGCCT
CGGGGCTGTCCGCGGGGGGACGGCTGCCTTCGGGGGGGACGGGGCAGGGC
GGGGTTCGGCTTCTGGCGTGTGACCGGCGGCTCTAGAGCCTCTGCTAACC
ATGTTCATGCCTTCTTCTTTTTCCTACAGCTCCTGGGCAACGTGCTGGTT
ATTGTGCTGTCTCATCATTTTGGCAAAGAATTCGAGCGGCCGCCAGCCGC
CACCATGGTGTGCTTTAGACTGTTTCCTGTGCCTGGTTCAGGGCTGGTCC
TGGTCTGTCTGGTGCTGGGGGCTGTCAGAAGCTATGCCTTGGAGCTGAAC
CTCACTGATAGTGAAAATGCCACTTGTCTGTATGCTAAGTGGCAGATGAA
CTTCACTGTGAGATATGAAACCACCAACAAGACTTACAAAACAGTGACCA
TCTCAGATCATGGAACTGTGACCTACAACGGCAGCATTTGTGGAGACGAC
CAGAACGGACCAAAAATCGCTGTCCAATTTGGGCCTGGATTCTCCTGGAT
TGCCAATTTCACTAAAGCTGCCTCCACATATTCAATTGACTCAGTGTCCT
TCTCCTACAACACTGGGGACAACACTACTTTCCCTGATGCTGAAGATAAG
GGAATCTTGACAGTGGATGAGCTGCTGGCTATCAGGATCCCTTTGAATGA
CCTGTTTAGGTGTAATTCACTGAGCACTCTGGAGAAGAACGACGTGGTGC
AGCACTACTGGGACGTGCTGGTGCAGGCCTTTGTGCAGAACGGCACTGTG
TCCACCAACGAATTCCTGTGTGATAAGGACAAAACTTCCACTGTGGCACC
TACAATTCACACTACTGTGCCTTCACCTACCACCACTCCAACTCCAAAGG
AAAAGCCTGAAGCAGGAACCTACTCTGTGAACAATGGCAATGATACCTGT
CTGTTGGCCACCATGGGCCTCCAACTGAACATTACTCAGGACAAGGTGGC
CTCAGTGATTAACATTAACCCCAACACTACCCACTCCACTGGCAGCTGTA
GATCACACACAGCCTTGCTCAGACTGAATAGCAGCACCATCAAGTATTTG
GATTTTGTGTTTGCAGTGAAGAATGAAAACAGGTTCTACCTGAAGGAAGT
CAACATCTCAATGTACCTGGTGAACGGCTCAGTGTTCAGCATTGCCAACA
ACAACCTCTCCTATTGGGACGCTCCACTGGGGAGCAGCTACATGTGTAAC
AAGGAACAGACTGTGTCAGTGTCAGGAGCCTTCCAGATTAACACCTTTGA
TCTGAGGGTCCAACCCTTTAATGTCACTCAAGGAAAGTATAGCACTGCCC
AGGAGTGCTCCCTGGATGATGACACCATTCTGATTCCAATCATTGTGGGT
GCAGGACTTTCTGGGCTTATTATTGTGATTGTGATTGCCTATGTGATTGG
CAGAAGGAAATCCTATGCAGGGTACCAAACTCTGTAAAAGGGCGAATTCC
AGCACACGCGTCCTAGGAGCTCGAGTACTACTGGCGGCCGTTACTAGTGG
ATCCGCGGTACAAGTAAGCATGCAAGCTTCGAGGACGGGGTGAACTACGC
CTGAATCAAGCTTATCGATAAATTCGAGCATCTTACCGCCATTTATTCCC
ATATTTGTTCTGTTTTTCTTGATTTGGGTATACATTTAAATGTTAATAAA
ACAAAATGGTGGGGCAATCATTTACATTTTTAGGGATATGTAATTACTAG
TTCAGGTGTATTGCCACAAGACAAACATGTTAAGAAACTTTCCCGTTATT
TACGCTCTGTTCCTGTTAATCAACCTCTGGATTACAAAATTTGTGAAAGA
TTGACTGATATTCTTAACTATGTTGCTCCTTTTACGCTGTGTGGATATGC
TGCTTTAATGCCTCTGTATCATGCTATTGCTTCCCGTACGGCTTTCGTTT
TCTCCTCCTTGTATAAATCCTGGTTGCTGTCTCTTTATGAGGAGTTGTGG
CCCGTTGTCCGTCAACGTGGCGTGGTGTGCTCTGTGTTTGCTGACGCAAC
CCCCACTGGCTGGGGCATTGCCACCACCTGTCAACTCCTTTCTGGGACTT
TCGCTTTCCCCCTCCCGATCGCCACGGCAGAACTCATCGCCGCCTGCCTT
GCCCGCTGCTGGACAGGGGCTAGGTTGCTGGGCACTGATAATTCCGTGGT
GTTGTCGGGGAAGGGCCTCGATACCGTCGATATCGATCCTGGCTAATAAA
GGAAATTTATTTTCATTGCAATAGTGTGTTGGAATTTTTTGTGTCTCTCA
CTCGGAAGGACATATGGGAGGGCAAATCATTTAAAACATCAGAATGAGTA
TTTGGTTTAGAGTTTGGCAACATATGCCCATATGCTGGCTGCCATGAACA
AAGGTTGGCTATAAAGAGGTCATCAGTATATGAAACAGCCCCCTGCTGTC
CATTCCTTATTCCATAGAAAAGCCTTGACTTGAGGTTAGATTTTTTTTAT
ATTTTGTTTTGTGTTATTTTTTTCTTTAACATCCCTAAAATTTTCCTTAC
ATGTTTTACTAGCCAGATTTTTCCTCCTCTCCTGACTACTCCCAGTCATA
GCTGTCCCTCTTCTCTTATGGAGATCGAAGCAATTCGTTGATCTGAATTT
CGACCACCCATAATAGATCTCCCATTACCCTGGTAGATAAGTAGCATGGC
GGGTTAATCATTAACTACAAGGAACCCCTAGTGATGGAGTTGGCCACTCC
CTCTCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGCAG
(SEQ ID NO: 12)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCTTGTAGTTAATGATTAACCCG
CCATGCTACTTATCTACCAGGGTAATGGGGATCCTCTAGAACTATAGCTA
GTCGACATTGATTATTGACTAGTTATTAATAGTAATCAATTACGGGGTCA
TTAGTTCATAGCCCATATATGGAGTTCCGCGTTACATAACTTACGGTAAA
TGGCCCGCCTGGCTGACCGCCCAACGACCCCCGCCCATTGACGTCAATAA
TGACGTATGTTCCCATAGTAACGCCAATAGGGACTTTCCATTGACGTCAA
TGGGTGGAGTATTTACGGTAAACTGCCCACTTGGCAGTACATCAAGTGTA
TCATATGCCAAGTACGCCCCCTATTGACGTCAATGACGGTAAATGGCCCG
CCTGGCATTATGCCCAGTACATGACCTTATGGGACTTTCCTACTTGGCAG
TACATCTACGTATTAGTCATCGCTATTACCATGGTCGAGGTGAGCCCCAC
GTTCTGCTTCACTCTCCCCATCTCCCCCCCCTCCCCACCCCCAATTTTGT
ATTTATTTATTTTTTAATTATTTTGTGCAGCGATGGGGGCGGGGGGGGGG
GGGGGGCGCGCGCCAGGCGGGGCGGGGCGGGGCGAGGGGCGGGGCGGGGC
GAGGCGGAGAGGTGCGGCGGCAGCCAATCAGAGCGGCGCGCTCCGAAAGT
TTCCTTTTATGGCGAGGCGGCGGCGGCGGCGGCCCTATAAAAAGCGAAGC
GCGCGGCGGGCGGGAGTCGCTGCGCGCTGCCTTCGCCCCGTGCCCCGCTC
CGCCGCCGCCTCGCGCCGCCCGCCCCGGCTCTGACTGACCGCGTTACTCC
CACAGGTGAGCGGGCGGGACGGCCCTTCTCCTCCGGGCTGTAATTAGCGC
TTGGTTTAATGACGGCTTGTTTCTTTTCTGTGGCTGCGTGAAAGCCTTGA
GGGGCTCCGGGAGGGCCCTTTGTGCGGGGGGAGCGGCTCGGGGGGTGCGT
GCGTGTGTGTGTGCGTGGGGAGCGCCGCGTGCGGCTCCGCGCTGCCCGGC
GGCTGTGAGCGCTGCGGGCGCGGCGCGGGGCTTTGTGCGCTCCGCAGTGT
GCGCGAGGGGAGCGCGGCCGGGGGCGGTGCCCCGCGGTGCGGGGGGGGCT
GCGAGGGGAACAAAGGCTGCGTGCGGGGTGTGTGCGTGGGGGGGTGAGCA
GGGGGTGTGGGCGCGTCGGTCGGGCTGCAACCCCCCCTGCACCCCCCTCC
CCGAGTTGCTGAGCACGGCCCGGCTTCGGGTGCGGGGCTCCGTACGGGGC
GTGGCGCGGGGCTCGCCGTGCCGGGCGGGGGGTGGCGGCAGGTGGGGGTG
CCGGGCGGGGCGGGGCCGCCTCGGGCCGGGGAGGGCTCGGGGGAGGGGCG
CGGCGGCCCCCGGAGCGCCGGCGGCTGTCGAGGCGCGGCGAGCCGCAGCC
ATTGCCTTTTATGGTAATCGTGCGAGAGGGCGCAGGGACTTCCTTTGTCC
CAAATCTGTGCGGAGCCGAAATCTGGGAGGCGCCGCCGCACCCCCTCTAG
CGGGCGCGGGGCGAAGCGGTGCGGCGCCGGCAGGAAGGAAATGGGCGGGG
AGGGCCTTCGTGCGTCGCCGCGCCGCCGTCCCCTTCTCCCTCTCCAGCCT
CGGGGCTGTCCGCGGGGGGACGGCTGCCTTCGGGGGGGACGGGGCAGGGC
GGGGTTCGGCTTCTGGCGTGTGACCGGCGGCTCTAGAGCCTCTGCTAACC
ATGTTCATGCCTTCTTCTTTTTCCTACAGCTCCTGGGCAACGTGCTGGTT
ATTGTGCTGTCTCATCATTTTGGCAAAGAATTCGAGCGGCCGCCAGCCGC
CACCATGGTCTGTTTTAGGCTGTTCCCTGTCCCTGGTTCAGGACTGGTCT
TAGTGTGTCTGGTGCTTGGAGCTGTCAGAAGCTATGCCCTGGAGCTGAAC
CTGACTGACTCAGAAAATGCCACTTGCCTGTATGCCAAGTGGCAGATGAA
CTTCACTGTCAGATATGAAACCACCAACAAGACCTATAAGACTGTGACCA
TCTCAGACCATGGCACTGTGACTTACAATGGGTCAATTTGTGGAGATGAC
CAGAATGGCCCTAAGATAGCTGTCCAGTTTGGTCCAGGATTCAGCTGGAT
TGCCAACTTCACCAAGGCAGCCAGCACCTACAGCATTGACTCTGTGTCCT
TCTCCTACAACACAGGAGACAACACCACTTTCCCTGATGCAGAGGACAAA
GGTATCCTGACTGTGGATGAGTTGCTGGCAATCAGGATCCCACTGAACGA
TCTGTTCAGGTGCAACTCACTGTCCACTCTGGAAAAGAATGATGTGGTGC
AGCACTATTGGGATGTGCTAGTCCAGGCCTTTGTCCAGAATGGGACTGTG
TCAACTAATGAGTTCCTGTGTGACAAGGACAAGACAAGCACTGTAGCCCC
CACTATCCATACCACAGTACCTAGCCCCACCACTACTCCAACCCCCAAGG
AGAAGCCTGAGGCTGGCACCTACTCAGTGAACAATGGGAATGACACCTGT
TTGCTGGCCACTATGGGACTCCAACTGAACATCACCCAGGACAAAGTGGC
CTCTGTGATCAATATCAATCCCAACACCACCCACAGCACTGGGTCCTGCA
GAAGCCACACTGCCCTCCTGAGGCTCAACTCATCAACTATCAAGTACTTG
GATTTTGTGTTTGCAGTGAAGAATGAGAACAGATTCTACCTCAAAGAGGT
CAACATTTCAATGTACCTGGTGAATGGGAGTGTGTTCTCCATTGCTAACA
ACAACCTGAGCTACTGGGATGCCCCTCTGGGCTCCTCATACATGTGCAAC
AAGGAACAGACTGTGAGTGTGTCAGGGGCCTTCCAGATCAACACTTTTGA
CCTGAGAGTGCAGCCCTTTAATGTGACACAGGGAAAGTACAGCACTGCTC
AGGAGTGCAGCCTGGATGATGACACTATCCTGATCCCTATCATTGTGGGG
GCAGGCCTGTCTGGACTCATTATTGTGATTGTGATTGCCTATGTGATAGG
GAGAAGGAAGTCTTATGCTGGATACCAGACCCTGTAAAAGGGCGAATTCC
AGCACACGCGTCCTAGGAGCTCGAGTACTACTGGCGGCCGTTACTAGTGG
ATCCGCGGTACAAGTAAGCATGCAAGCTTCGAGGACGGGGTGAACTACGC
CTGAATCAAGCTTATCGATAAATTCGAGCATCTTACCGCCATTTATTCCC
ATATTTGTTCTGTTTTTCTTGATTTGGGTATACATTTAAATGTTAATAAA
ACAAAATGGTGGGGCAATCATTTACATTTTTAGGGATATGTAATTACTAG
TTCAGGTGTATTGCCACAAGACAAACATGTTAAGAAACTTTCCCGTTATT
TACGCTCTGTTCCTGTTAATCAACCTCTGGATTACAAAATTTGTGAAAGA
TTGACTGATATTCTTAACTATGTTGCTCCTTTTACGCTGTGTGGATATGC
TGCTTTAATGCCTCTGTATCATGCTATTGCTTCCCGTACGGCTTTCGTTT
TCTCCTCCTTGTATAAATCCTGGTTGCTGTCTCTTTATGAGGAGTTGTGG
CCCGTTGTCCGTCAACGTGGCGTGGTGTGCTCTGTGTTTGCTGACGCAAC
CCCCACTGGCTGGGGCATTGCCACCACCTGTCAACTCCTTTCTGGGACTT
TCGCTTTCCCCCTCCCGATCGCCACGGCAGAACTCATCGCCGCCTGCCTT
GCCCGCTGCTGGACAGGGGCTAGGTTGCTGGGCACTGATAATTCCGTGGT
GTTGTCGGGGAAGGGCCTCGATACCGTCGATATCGATCCTGGCTAATAAA
GGAAATTTATTTTCATTGCAATAGTGTGTTGGAATTTTTTGTGTCTCTCA
CTCGGAAGGACATATGGGAGGGCAAATCATTTAAAACATCAGAATGAGTA
TTTGGTTTAGAGTTTGGCAACATATGCCCATATGCTGGCTGCCATGAACA
AAGGTTGGCTATAAAGAGGTCATCAGTATATGAAACAGCCCCCTGCTGTC
CATTCCTTATTCCATAGAAAAGCCTTGACTTGAGGTTAGATTTTTTTTAT
ATTTTGTTTTGTGTTATTTTTTTCTTTAACATCCCTAAAATTTTCCTTAC
ATGTTTTACTAGCCAGATTTTTCCTCCTCTCCTGACTACTCCCAGTCATA
GCTGTCCCTCTTCTCTTATGGAGATCGAAGCAATTCGTTGATCTGAATTT
CGACCACCCATAATAGATCTCCCATTACCCTGGTAGATAAGTAGCATGGC
GGGTTAATCATTAACTACAAGGAACCCCTAGTGATGGAGTTGGCCACTCC
CTCTCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGCAG.

In an embodiment, the vector is an adeno-associated virus (AAV) vector. In an embodiment, the expression cassette comprises ITR sequences selected from SEQ ID NOs: 13 and 14.

(SEQ ID NO: 13)
CTGCGCGCTCGCTCGCTCACTGAGGCCGCCCGGGCAAAGCCCGGGCGTCG
GGCGACCTTTGGTCGCCCGGCCTCAGTGAGCGAGCGAGCGCGCAGAGAGG
GAGTGGCCAACTCCATCACTAGGGGTTCCT
(SEQ ID NO: 14)
AGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCG
GGCGGCCTCAGTGAGCGAGCGAGCGCGCAG

In related embodiments, the disclosure provides gene therapy vectors comprising an expression cassette disclosed herein. Generally, the gene therapy vectors described herein comprise an expression cassette comprising a polynucleotide encoding one or more isoforms of lysosome-associated membrane protein 2 (LAMP-2), that allows for the expression of LAMP-2 to partially or wholly rectify deficient LAMP-2 protein expression levels and/or autophagic flux in a subject in need thereof (e.g., a subject having Danon disease or another disorder characterized by deficient autophagic flux at least in part due to deficient LAMP-2 expression).

LAMP-2A protein sequence
(SEQ ID NO: 15)
MVCFRLFPVPGSGLVLVCLVLGAVRSYALELNLTDSENATCLYAKWQMNF
TVRYETTNKTYKTVTISDHGTVTYNGSICGDDQNGPKIAVQFGPGFSWIA
NFTKAASTYSIDSVSFSYNTGDNTTFPDAEDKGILTVDELLAIRIPLNDL
FRCNSLSTLEKNDVVQHYWDVLVQAFVQNGTVSTNEFLCDKDKTSTVAPT
IHTTVPSPTTTPTPKEKPEAGTYSVNNGNDTCLLATMGLQLNITQDKVAS
VININPNTTHSTGSCRSHTALLRLNSSTIKYLDFVFAVKNENRFYLKEVN
ISMYLVNGSVFSIANNNLSYWDAPLGSSYMCNKEQTVSVSGAFQINTFDL
RVQPFNVTQGKYSTAQDCSADDDNFLVPIAVGAALAGVLILVLLAYFIGL
KHHHAGYEQF
LAMP-2B protein sequence
(SEQ ID NO: 16)
MVCFRLFPVPGSGLVLVCLVLGAVRSYALELNLTDSENATCLYAKWQMNF
TVRYETTNKTYKTVTISDHGTVTYNGSICGDDQNGPKIAVQFGPGFSWIA
NFTKAASTYSIDSVSFSYNTGDNTTFPDAEDKGILTVDELLAIRIPLNDL
FRCNSLSTLEKNDVVQHYWDVLVQAFVQNGTVSTNEFLCDKDKTSTVAPT
IHTTVPSPTTTPTPKEKPEAGTYSVNNGNDTCLLATMGLQLNITQDKVAS
VININPNTTHSTGSCRSHTALLRLNSSTIKYLDFVFAVKNENRFYLKEVN
ISMYLVNGSVFSIANNNLSYWDAPLGSSYMCNKEQTVSVSGAFQINTFDL
RVQPFNVTQGKYSTAQECSLDDDTILIPIIVGAGLSGLIIVIVIAYVIGR
RKSYAGYQTL
LAMP-2C protein sequence
(SEQ ID NO: 17)
MVCFRLFPVPGSGLVLVCLVLGAVRSYALELNLTDSENATCLYAKWQMNF
TVRYETTNKTYKTVTISDHGTVTYNGSICGDDQNGPKIAVQFGPGFSWIA
NFTKAASTYSIDSVSFSYNTGDNTTFPDAEDKGILTVDELLAIRIPLNDL
FRCNSLSTLEKNDVVQHYWDVLVQAFVQNGTVSTNEFLCDKDKTSTVAPT
IHTTVPSPTTTPTPKEKPEAGTYSVNNGNDTCLLATMGLQLNITQDKVAS
VININPNTTHSTGSCRSHTALLRLNSSTIKYLDFVFAVKNENRFYLKEVN
ISMYLVNGSVFSIANNNLSYWDAPLGSSYMCNKEQTVSVSGAFQINTFDL
RVQPFNVTQGKYSTAEECSADSDLNFLIPVAVGVALGFLIIVVFISYMIG
RRKSRTGYQSV

In particular embodiments, the expression cassette comprises a polynucleotide sequence encoding LAMP-2 disclosed herein, e.g., SEQ ID NOs: 15-17 or a sequence having at least 90%, at least 95%, at least 98%, or at least 99% identity to any of SEQ ID NOs: 15-17. The gene therapy vectors can be viral or non-viral vectors. Illustrative non-viral vectors include, e.g., naked DNA, cationic liposome complexes, cationic polymer complexes, cationic liposome-polymer complexes, and exosomes. Examples of viral vector include, but are not limited, to adenoviral, retroviral, lentiviral, herpesvirus and adeno-associated virus (AAV) vectors.

In some embodiments, the expression cassette comprising a polynucleotide sequence encoding one or more, two or more, or all three of SEQ ID NOs: 15-17. In some embodiments, the polynucleotide sequence comprising the native introns of the LAMP-2 gene, enabling expression of more than one isoform in the same cell using one vector. In some embodiments, artificial introns, splice acceptors, and/or splice donors are using to optimize the length of the polynucleotide and/or optimize the ratio of isoforms expressed by the polynucleotide encoding two or more, or all three of SEQ ID NOs: 15-17.

In some embodiments, the expression cassette, AAV capsid gene, and/or helper genes are delivered to cells using transduction, transfection, electroporation, lipofection, and any other methods known in the art. In some embodiments, the expression cassette, AAV capsid gene, and/or helper genes are delivered in a liposome or a lipid nanoparticle (LNP). The expression cassette, AAV capsid gene, and/or helper genes may be provide as DNA, e.g. on one or more plasmids, bacmids, or other DNA molecules. In some embodiments, expression cassette, AAV capsid gene, and/or helper genes are delivery as RNA molecules. In some embodiments, the RNA molecules comprise one or more mRNA molecules, e.g., one or more in vitro transcribed mRNA molecules. In some embodiments, the mRNA molecules are modified mRNA molecules. Illustrative modifications include lock nucleic acids, phoshothiolate linkages, and modified nucleosides (e.g. pseudouridine, 5-methylcytosine, or 5-methylcytidine). In some embodiments, the modified mRNA comprises a cap, e.g. an ARCA cap. The expression cassette, AAV capsid gene, and/or helper genes may be delivered in vitro or in vivo. In some embodiments, the AAV capsid gene comprises one or more of an AAV9 capsid gene and an AAVrh74 capsid gene.

Gene delivery viral vectors useful in the practice of the present invention can be constructed utilizing methodologies well known in the art of molecular biology. Typically, viral vectors carrying transgenes are assembled from polynucleotides encoding the transgene, suitable regulatory elements and elements necessary for production of viral proteins, which mediate cell transduction. Such recombinant viruses may be produced by techniques known in the art, e.g., by transfecting packaging cells or by transient transfection with helper plasmids or viruses. Typical examples of virus packaging cells include but are not limited to HeLa cells, SF9 cells (optionally with a baculovirus helper vector), 293 cells, etc. A Herpesvirus-based system can be used to produce AAV vectors, as described in US20170218395A1. Detailed protocols for producing such replication-defective recombinant viruses may be found for instance in WO95/14785, WO96/22378, U.S. Pat. Nos. 5,882,877, 6,013,516, 4,861,719, 5,278,056 and WO94/19478, the complete contents of each of which is hereby incorporated by reference.

AAV is a 4.7 kb, single stranded DNA virus. Recombinant vectors based on AAV are associated with excellent clinical safety, since wild-type AAV is nonpathogenic and has no etiologic association with any known diseases. In addition, AAV offers the capability for highly efficient gene delivery and sustained transgene expression in numerous tissues. By an “AAV vector” is meant a vector derived from an adeno-associated virus serotype, including without limitation, AAV1, AAV2, AAV3, AAV4, AAV5, AAV6, AAV7, AAV8, AAV9, AAV10, AAVrh.10, AAVrh74, etc. AAV vectors can have one or more of the AAV wild-type genes deleted in whole or part, e.g., the rep and/or cap genes, but retain functional flanking inverted terminal repeat (ITR) sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g. by the insertion, deletion or substitution of nucleotides, as long as the sequences provide for functional rescue, replication and packaging. AAV vectors may comprise other modifications, including but not limited to one or more modified capsid proteins (e.g., VP1, VP2 and/or VP3). For example, a capsid protein may be modified to alter tropism and/or reduce immunogenicity. AAV expression vectors are constructed using known techniques to at least provide as operatively linked components in the direction of transcription, control elements including a transcriptional initiation region, the DNA of interest (i.e. the LAMP-2 gene) and a transcriptional termination region.

Adeno-associated virus (AAV) is single stranded DNA virus. The AAV genome is built of single-stranded deoxyribonucleic acid (ssDNA), either positive- or negative-sensed, which is about 4.7 kilobase long. The genome comprises inverted terminal repeats (ITRs) at both ends of the DNA strand, and two open reading frames (ORFs): rep and cap. The first, rep, is composed of four overlapping genes encoding Rep proteins required for the AAV life cycle, and the second, cap, encodes three capsid proteins: VP1, VP2 and VP3. The cap gene is expressed as a messenger RNA (mRNA) from the p40 promoter of AAV. The mRNA is alternatively spliced into 2.3 kb and 2.6 kb transcripts, with the 2.3 kb transcript being more abundant. VP1 is expressed only from the 2.6 kb transcript and the VP1 protein is 87 kilodaltons (kDa) in molecular weight. VP2 is expressed from an open reading frame that begins with an ACG codon, rather than a canonical AUG codon, due to the presence of an optimal Kozak sequence for translation initiation. VP2 is 72 kDa. VP3, only 62 kDa, is expressed from the ATG sequence presence in the 2.3 kb transcript, as well as the 2.6 kb transcript. The relative abundances of VP1:VP2:VP3 are 1:1:10. VP1, VP2, and VP3 interact together to form a capsid of an icosahedral symmetry.

Recombinant vectors based on AAV are associated with excellent clinical safety, since wild-type AAV is nonpathogenic and has no etiologic association with any known diseases. In addition, AAV offers the capability for highly efficient gene delivery and sustained transgene expression in numerous tissues. Various serotypes of AAV are known, including, AAV1, AAV2, AAV3, AAV4, AAV5, AAV6, AAV7, AAV8, AAV9, AAV10, AAVrh.10, AAVrh74, etc. AAV vectors can have one or more of the AAV wild-type genes deleted in whole or part, e.g., the rep and/or cap genes, but retain functional flanking inverted terminal repeat (ITR) sequences. The serotype of a recombinant AAV vector is determined by its capsid. International Patent Publication No. WO2003042397A2 discloses various capsid sequences including those of AAV1, AAV2, AAV3, AAV8, AAV9, and AAVrh10. International Patent Publication No. WO2013078316A1 discloses the polypeptide sequence of the VP1 from AAVrh74. Numerous diverse naturally occurring or genetically modified AAV capsid sequences are known in the art.

The present disclosure also provides pharmaceutical compositions comprising an expression cassette or vector (e.g., gene therapy vector) disclosed herein and one or more pharmaceutically acceptable carriers, diluents or excipients. In particular embodiments, the pharmaceutical composition comprises an AAV vector comprising an expression cassette disclosed herein, e.g., wherein the expression cassette comprises a codon-optimized transgene encoding LAMP-2B, e.g., any of SEQ ID NOs: 7-9. Provided are pharmaceutical compositions, e.g., for use in preventing or treating a disorder characterized by deficient autophagic flux (e.g., Danon disease) which comprises a therapeutically effective amount of a vector that comprises a nucleic acid sequence of a polynucleotide that encodes one or more isoforms of LAMP-2.

The pharmaceutical compositions that contain the expression cassette or vector may be in any form that is suitable for the selected mode of administration, for example, for intraventricular, intramyocardial, intracoronary, intravenous, intra-arterial, intra-renal, intraurethral, epidural or intramuscular administration. The gene therapy vector comprising a polynucleotide encoding one or more LAMP-2 isoforms can be administered, as sole active agent, or in combination with other active agents, in a unit administration form, as a mixture with conventional pharmaceutical supports, to animals and human beings. In some embodiments, the pharmaceutical composition comprises cells transduced ex vivo with any of the gene therapy vectors of the disclosure.

In various embodiments, the pharmaceutical compositions contain vehicles (e.g., carriers, diluents and excipients) that are pharmaceutically acceptable for a formulation capable of being injected. These may be in particular isotonic, sterile, saline solutions (monosodium or disodium phosphate, sodium, potassium, calcium or magnesium chloride and the like or mixtures of such salts). Illustrative pharmaceutical forms suitable for injectable use include, e.g., sterile aqueous solutions or dispersions; formulations including sesame oil, peanut oil or aqueous propylene glycol; and sterile powders for the extemporaneous preparation of sterile injectable solutions or dispersions.

In another aspect, the disclosure provides methods of preventing, mitigating, ameliorating, reducing, inhibiting, eliminating and/or reversing one or more symptoms of Danon disease or another autophagy disorder in a subject in need thereof, comprising administering to the subject a gene therapy vector of the disclosure. The term “Danon disease” refers to an X-linked dominant skeletal and cardiac muscle disorder with multisystem clinical manifestations. Danon disease mutations lead to an absence of lysosome-associated membrane protein 2 (LAMP-2) protein expression. Major clinical features include skeletal and cardiac myopathy, cardiac conduction abnormalities, cognitive difficulties, and retinal disease. Men are typically affected earlier and more severely than women.

In an embodiment, the vector is administered via a route selected from the group consisting of parenteral, intravenous, intra-arterial, intracardiac, intracoronary, intramyocardial, intrarenal, intraurethral, epidural, and intramuscular. In an embodiment, the vector is administered multiple times. In an embodiment, the vector is administered by intramuscular injection of the vector. In an embodiment, the vector is administered by injection of the vector into skeletal muscle. In an embodiment, the expression cassette comprises a muscle-specific promoter, optionally a muscle creatine kinase (MCK) promoter or a MCK/SV40 hybrid promoter as described in Takeshita et al. Muscle creatine kinase/SV40 hybrid promoter for muscle-targeted long-term transgene expression. Int J Mol Med. 2007 February; 19(2):309-15. In an embodiment, the vector is administered by intracardiac injection.

In an embodiment, the disclosure provides a method of treating a disease or disorder, optionally Danon disease, in a subject in need thereof, comprising contacting cells with a gene therapy vector according to the present disclosure and administering the cells to the subject. In an embodiment, the cells are stem cells, optionally pluripotent stem cells. In an embodiment, the stem cells are capable of differentiation into cardiac tissue. In an embodiment, the stem cells are capable of differentiation into muscle tissue, e.g., cardiac muscle tissue and/or skeletal muscle tissue. In an embodiment, the stem cells are autologous. In an embodiment, the stem cells are induced pluripotent stem cells (iPSCs).

In an embodiment, the autophagy disorder is selected from the group consisting of end-stage heart failure, myocardial infarction, drug toxicities, diabetes, end-stage renal failure, and aging. In an embodiment, the subject is a mammal, e.g., a human. In an embodiment, the subject is exhibiting symptoms of Danon disease or another autophagy disorder. In an embodiment, the subject has been identified as having reduced or non-detectable LAMP-2 expression. In an embodiment, the subject has been identified as having a mutated LAMP-2 gene.

Subjects/patients amenable to treatment using the methods described herein include individuals at risk of a disease or disorder characterized by insufficient autophagic flux (e.g., Danon disease as well as other known disorders of autophagy including, but not limited to, systolic and diastolic heart failure, myocardial infarction, drug toxicities (for example, anthracyclines, chloroquine, and its derivatives), diabetes, end-stage renal disease, and aging) but not showing symptoms, as well as subjects presently showing symptoms. Such subject may have been identified as having a mutated LAMP-2 gene or as having reduced or non-detectable levels of LAMP-2 expression.

In some embodiments, the subject is exhibiting symptoms of a disease or disorder characterized by insufficient autophagic flux (e.g., Danon disease as well as other known disorders of autophagy including, but not limited to, systolic and diastolic heart failure, myocardial infarction, drug toxicities, diabetes, end-stage renal disease, and aging). The symptoms may be actively manifesting, or may be suppressed or controlled (e.g., by medication) or in remission. The subject may or may not have been diagnosed with the disorder, e.g., by a qualified physician.

### Definitions

The terms “lysosome-associated membrane protein 2” and “LAMP-2” interchangeably refer to nucleic acids and polypeptide polymorphic variants, alleles, mutants, and interspecies homologs that: (1) have an amino acid sequence that has greater than about 90% amino acid sequence identity, for example, 91%, 92%, 93%, 94%, 95%, 96%, 97%, 98% or 99% or greater amino acid sequence identity, preferably over a region of at least about 25, 50, 100, 200, 300, 400, or more amino acids, or over the full-length, to an amino acid sequence encoded by a LAMP-2 nucleic acid (see, e.g., GenBank Accession Nos. NM_002294.2 (isoform A). NM_013995.2 (isoform B), NM_001122606.1 (isoform C)) or to an amino acid sequence of a LAMP-2 polypeptide (see e.g., GenBank Accession Nos. NP_002285.1 (isoform A), NP_054701.1 (isoform B), NP_001116078.1 (isoform C)); (2) bind to antibodies, e.g., polyclonal antibodies, raised against an immunogen comprising an amino acid sequence of a LAMP-2 polypeptide (e.g., LAMP-2 polypeptides described herein); or an amino acid sequence encoded by a LAMP-2 nucleic acid (e.g., LAMP-2 polynucleotides described herein), and conservatively modified variants thereof; (3) specifically hybridize under stringent hybridization conditions to an anti-sense strand corresponding to a nucleic acid sequence encoding a LAMP-2 protein, and conservatively modified variants thereof; (4) have a nucleic acid sequence that has greater than about 90%, preferably greater than about 91%, 92%, 93%, 94%, 95%, 96%, 97%, 98%, 99%, or higher nucleotide sequence identity, preferably over a region of at least about 25, 50, 100, 200, 500, 1000, 2000 or more nucleotides, or over the full-length, to a LAMP-2 nucleic acid (e.g., LAMP-2 polynucleotides, as described herein, and LAMP-2 polynucleotides that encode LAMP-2 polypeptides, as described herein).

The terms “identical” or percent “identity,” in the context of two or more nucleic acids or polypeptide sequences, refer to two or more sequences or subsequences that are the same or have a specified percentage of amino acid residues or nucleotides that are the same (i.e., share at least about 80% identity, for example, at least about 85%, 86%, 87%, 88%, 89%, 90%, 91%, 92%, 93%, 94%, 95%, 96%, 97%, 98%, 99% identity over a specified region to a reference sequence, e.g., LAMP-2 polynucleotide or polypeptide sequence as described herein, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, for example, over a region that is 50, 100, 200, 300, 400 amino acids or nucleotides in length, or over the full-length of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to LAMP-2 nucleic acids and proteins, the BLAST and BLAST 2.0 algorithms and the default parameters are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., eds., Current Protocols in Molecular Biology (1995 supplement)). Examples of algorithms that are suitable for determining percent sequence identity and sequence similarity are the BLAST and BLAST 2.0 algorithms, which are described in Altschul et al., J. Mol. Biol. 215:403-410 (1990) and Altschul et al., Nucleic Acids Res. 25:3389-3402 (1977), respectively. Software for performing BLAST analyses is publicly available through the National Center for Biotechnology Information (on the worldwide web at ncbi.nlm.nih.gov/).

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions. Yet another indication that two nucleic acid sequences are substantially identical is that the same primers can be used to amplify the sequence.

As used herein, “administering” refers to local and systemic administration, e.g., including enteral, parenteral, pulmonary, and topical/transdermal administration. Routes of administration for compounds (e.g., polynucleotide encoding one or more LAMP-2 isoforms) that find use in the methods described herein include, e.g., oral (per os (P.O.)) administration, nasal or inhalation administration, administration as a suppository, topical contact, transdermal delivery (e.g., via a transdermal patch), intrathecal (IT) administration, intravenous (“iv”) administration, intraperitoneal (“ip”) administration, intramuscular (“im”) administration, intralesional administration, or subcutaneous (“sc”) administration, or the implantation of a slow-release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intraarterial, intrarenal, intraurethral, intracardiac, intracoronary, intramyocardial, intradermal, epidural, subcutaneous, intraperitoneal, intraventricular, ionophoretic and intracranial. Other modes of delivery include, but are not limited to, the use of liposomal formulations, intravenous infusion, transdermal patches, etc. In some embodiments, the dose of rAAV gene therapy vector administered is about 1E+11 vector genomes (vg)/kg to about 1E+12 vg/kg, about 1E+12 vg/kg to about 2E+12 vg/kg, about 2E+12 vg/kg to about 3E+12 vg/kg, about 3E+12 vg/kg to about 3E+13 vg/kg, or about 3E+13 vg/kg to about 3E+14 vg/kg. In some embodiments, the dose of rAAV gene therapy vector administered is about 3E+12 vg/kg to about 3E+14 vg/kg.

The terms “systemic administration” and “systemically administered” refer to a method of administering a compound or composition to a mammal so that the compound or composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

The term “co-administering” or “concurrent administration”, when used, for example with respect to the compounds (e.g., LAMP-2 polynucleotides) and/or analogs thereof and another active agent, refers to administration of the compound and/or analogs and the active agent such that both can simultaneously achieve a physiological effect. The two agents, however, need not be administered together. In certain embodiments, administration of one agent can precede administration of the other. Simultaneous physiological effect need not necessarily require presence of both agents in the circulation at the same time. However, in certain embodiments, co-administering typically results in both agents being simultaneously present in the body (e.g., in the plasma) at a significant fraction (e.g., 20% or greater, e.g., 30% or 40% or greater, e.g., 50% or 60% or greater, e.g., 70% or 80% or 90% or greater) of their maximum serum concentration for any given dose.

The term “effective amount” or “pharmaceutically effective amount” refer to the amount and/or dosage, and/or dosage regime of one or more compounds (e.g., gene therapy vectors) necessary to bring about the desired result e.g., increased expression of one or more LAMP-2 isoforms in an amount sufficient to reduce the ultimate severity of a disease characterized by impaired or deficient autophagy (e.g., Danon disease). In some embodiments, the effective amount is about 1E+11 vg/kg to about 1E+12 vg/kg, about 1E+12 vg/kg to about 2E+12 vg/kg, about 2E+12 vg/kg to about 3E+12 vg/kg, about 3E+12 vg/kg to about 3E+13 vg/kg, or about 3E+13 vg/kg to about 3E+14 vg/kg of rAAV gene therapy vector. In some embodiments, the effective amount is about 3E+12 vg/kg to about 3E+14 vg/kg of rAAV gene therapy vector.

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

As used herein, the terms “treating” and “treatment” refer to delaying the onset of, retarding or reversing the progress of, reducing the severity of, or alleviating or preventing either the disease or condition to which the term applies, or one or more symptoms of such disease or condition. The terms “treating” and “treatment” also include preventing, mitigating, ameliorating, reducing, inhibiting, eliminating and/or reversing one or more symptoms of the disease or condition.

The term “mitigating” refers to reduction or elimination of one or more symptoms of that pathology or disease, and/or a reduction in the rate or delay of onset or severity of one or more symptoms of that pathology or disease, and/or the prevention of that pathology or disease. In certain embodiments, the reduction or elimination of one or more symptoms of pathology or disease can include, e.g., measurable and sustained increase in the expression levels of one or more isoforms of LAMP-2.

As used herein, the phrase “consisting essentially of refers to the genera or species of active pharmaceutical agents recited in a method or composition, and further can include other agents that, on their own do not have substantial activity for the recited indication or purpose.

The terms “subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig) and agricultural mammals (e.g., equine, bovine, porcine, ovine). In various embodiments, the subject can be a human (e.g., adult male, adult female, adolescent male, adolescent female, male child, female child).

The terms “gene transfer” or “gene delivery” refer to methods or systems for reliably inserting foreign DNA into host cells. Such methods can result in transient expression of non-integrated transferred DNA, extrachromosomal replication and expression of transferred replicons (e.g. episomes), or integration of transferred genetic material into the genomic DNA of host cells.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication or reverse transcription in a cell, or may include sequences sufficient to allow integration into host cell DNA. “vectors” include gene therapy vectors. As used herein, the term “gene therapy vector” refers to a vector capable of use in performing gene therapy, e.g., delivering a polynucleotide sequence encoding a therapeutic polypeptide to a subject. Gene therapy vectors may comprise a nucleic acid molecule (“transgene”) encoding a therapeutically active polypeptide, e.g., a LAMP-2B or other gene useful for replacement gene therapy when introduced into a subject. Useful vectors include, but are not limited to, viral vectors.

As used herein, the term “expression cassette” refers to a DNA segment that is capable in an appropriate setting of driving the expression of a polynucleotide (a “transgene”) encoding a therapeutically active polypeptide (e.g., LAMP-2B) that is incorporated in said expression cassette. When introduced into a host cell, an expression cassette inter alia is capable of directing the cell's machinery to transcribe the transgene into RNA, which is then usually further processed and finally translated into the therapeutically active polypeptide. The expression cassette can be comprised in a gene therapy vector. Generally, the term expression cassette excludes polynucleotide sequences 5′ to the 5′ ITR and 3′ to the 3′ ITR.

All patents, patent publications, and other publications referenced and identified in the present specification are individually and expressly incorporated herein by reference in their entirety for all purposes.

### EXAMPLES

### Example 1: Pre-Clinical and Clinical Evaluation of AAVrh74-LAMP-2B

A plasmid vector including the gene expression cassette as depicted in FIG. 1 is generated. The transgene is modified to encode LAM2B-HA-FLAG, so that the protein may be detected using either anti-HA or anti-FLAG antibodies. AAVrh74-LAMP2B viral vector is generated using a three-plasmid, helper virus-free system to generate recombinant AAV particles containing serotype rh74 capsid proteins and viral genomes that have AAV2 ITRs flanking a human LAMP-2B expression cassette. The viral vector is tested in non-human primates.

Pharmacology and toxicology studies are conducted in LAMP-2B^{-/-} and wild-type mice. Based on the preclinical safety and efficacy data observed in mice and non-human primate studies, clinical studies in patients with Danon disease are performed.

### Example 2: DNA, RNA, and Protein Expression in Non-Human Primates Following Intravenous Administration of 1×10¹³ vg/kg AAV9.LAMP2B and AAVrh74.LAMP2B

Non-human primate studies of AAV9 versus AAVrh74 vectors were performed in paired male and female African Green Monkeys (AGM). Subjects received either AAV9.LAMP2B-HA-Flag or AAVrh74.LAMP2B-HA-Flag. “AAV9.LAMP2B-HA-Flag” is an AAV9 serotype adeno-associated virus vector encoding LAMP2B C-terminally fused to an HA-Flag tag. “AAVrh74.LAMP2B-HA-Flag” is an AAVrh74 serotype adeno-associated virus vector encoding LAMP2B C-terminally fused to an HA-Flag tag. One subject was given vehicle control. The vectors were administered by intravenous injection of 2 mL of 1.85×10¹³ vector genomes (vg)/mL as determined by quantitative polymerase chain reaction (qPCR) using a plasmid containing the WPRE sequence to generate a reference curve. This injection achieved the target dose of vector, which was about 1.0×10¹³ vg/kg. Due to lower body weight, female subjects received about 1.2×10¹³ vg/kg of their respective vectors. This experiment is summarized in Table 2.

TABLE 2

| Animal | Sex | Treatment | Weight (kg) | Dose (vg/kg) |
|---|---|---|---|---|
| B059 | M | Vehicle buffer | 3.88 | n/a |
| A991 | F | AAV9 | 2.97 | 1.2 × 10¹³ |
| A602 | M | AAV9 | 3.56 | 1.0 × 10¹³ |
| A710 | F | AAVrh74 | 3.27 | 1.2 × 10¹³ |
| A981 | M | AAVrh74 | 3.69 | 1.0 × 10¹³ |

Subjects were humanely sacrificed two months after injection and tissues were collected for DNA, RNA, and protein analysis. The following tissues were examined: heart (left atrium, right atrium, left ventricle and right ventricle); skeletal muscle (quadricep and gastrocnemius); liver (left, right, middle and quadrate lobes); brain (frontal lobe, parietal lobe, temporal lobe, occipital lobe, cortex, hippocampus, medulla, and cerebellum); and gonads.

### Vector DNA—Quantitative PCR

DNA was extracted from frozen tissues using Qiagen DNeasy® kit. DNA purity (A260/A280) and concentration were evaluated on a NanoDrop One™ spectrophotometer (Thermo). Quantitative PCR (qPCR) was performed on 20 ng DNA using TaqMan Universal Master Mix II (Thermo, 4440038) on a real-time PCR system (QuantStudio5, Thermo) using the following primers/probes:

### WPRE (Cassette):

Forward primer:
(SEQ ID NO: 30)
5′-ATCATGCTATTGCTTCCCGTA-3′
Reverse primer:
(SEQ ID NO: 31)
5′-GGGCCACAACTCCTCATAAA-3′
Probe:
(SEQ ID NO: 32)
5′-CCTCCTTGTATAAATCCTGGTTGCTGTCT-3′

### RNaseP (Housekeeping Gene, Thermo)

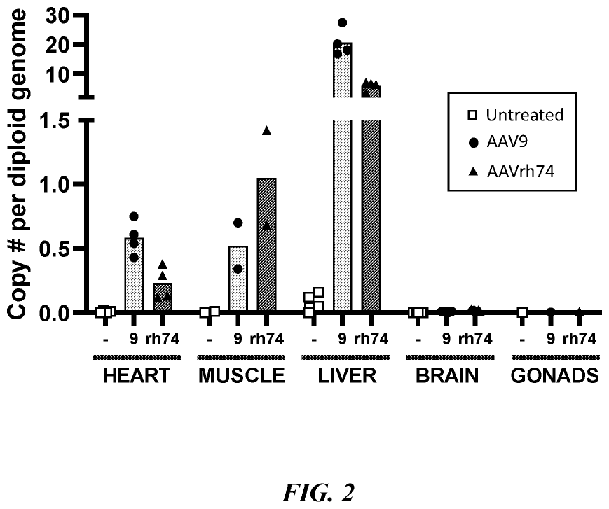

A standard curve was generated using plasmid DNA containing the WPRE sequence. FIG. 2 shows a bar graph of vector DNA quantification in organs most affected in Danon disease by qPCR.

LAMP2B mRNA—Quantitative RT-PCR

RNA was extracted from heart and muscle tissues using the RNeasy Fibrous Tissue kit (Qiagen), and from liver and brain using RNeasy Lipid Tissue kit (Qiagen). Purity (A260/A280) and concentration were determined on a NanoDrop One spectrophotometer. RNA was converted to cDNA using the Superscript IV VILO master mix (Thermo). qPCR was performed on 10 ng of RNA in TaqMan Universal Master Mix II (Thermo) on a real-time PCR system (QuantStudio5, Thermo) using the following primers/probes:

### Wpre (Cassette):

Forward primer:
(SEQ ID NO: 33)
5′-ATCATGCTATTGCTTCCCGTA-3′
Reverse primer:
(SEQ ID NO: 34)
5′-GGGCCACAACTCCTCATAAA-3′
Probe:
(SEQ ID NO: 35)
5′-CCTCCTTGTATAAATCCTGGTTGCTGTCT-3′

### HPRT-1 (Housekeeping Gene, Thermo)

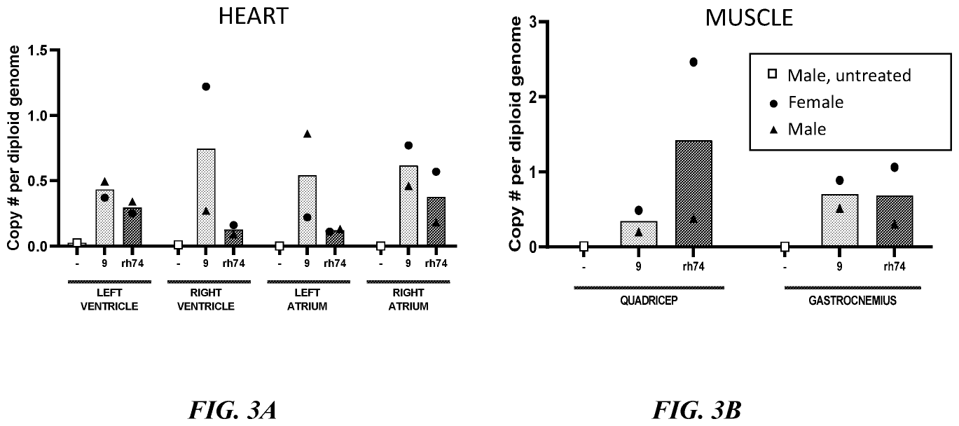

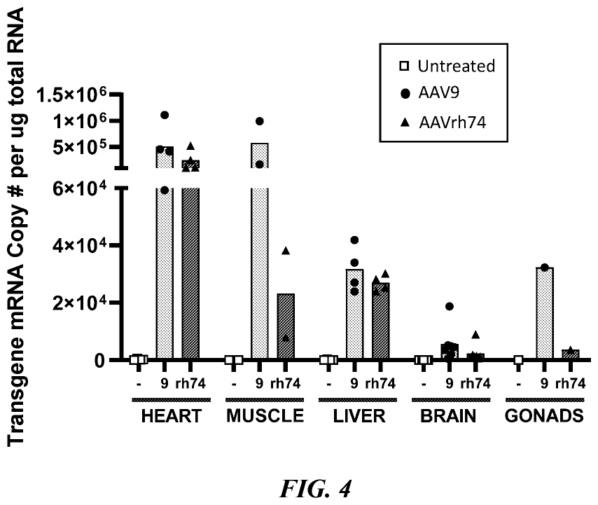

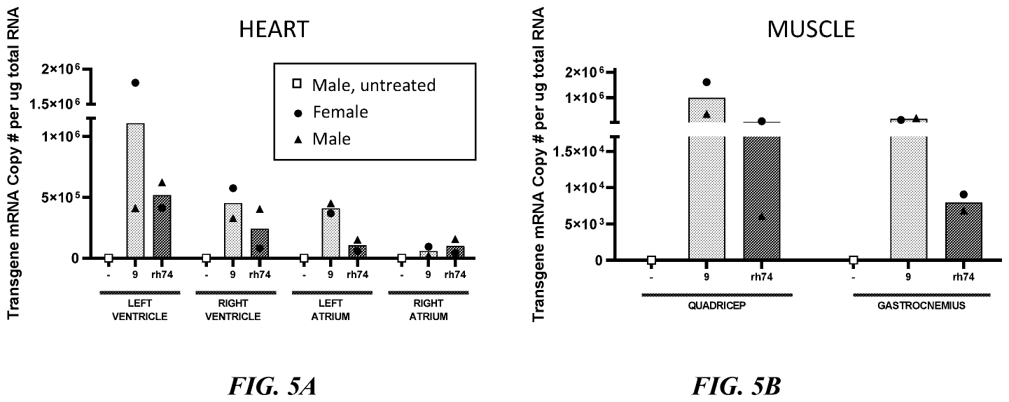

A standard curve was generated using plasmid DNA containing the WPRE sequence. FIG. 3A shows a bar graph of vector DNA quantification in regions of the heart by qPCR. FIG. 3B shows a bar graph of vector DNA quantification in muscles by qPCR. FIG. 4 shows a bar graph of mRNA quantification in organs most affected by Danon disease by RT-qPCR. FIG. 5A shows a bar graph of mRNA quantification in regions of the heart by RT-qPCR. FIG. 5B shows a bar graph of mRNA quantification in muscles by RT-qPCR.

LAMP2B mRNA—RNAscope

5 mm tissue cubes fixed in 10% neutral buffered formalin, embedded in paraffin and sectioned. Transgene mRNA was detected using WPRE-O3 ZZ probe (ACD) with RNAscope 2.5 LS RED. Semi-quantitative visual assessment of one section from each tissue was performed with cells with ≥1 dot per cell considered positive. The percentage of cells positive were binned into five categories: 0%, 1-25%, 26-50%, 51-75% or 100%.

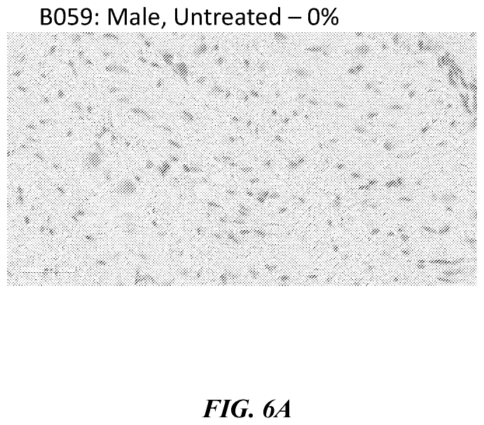

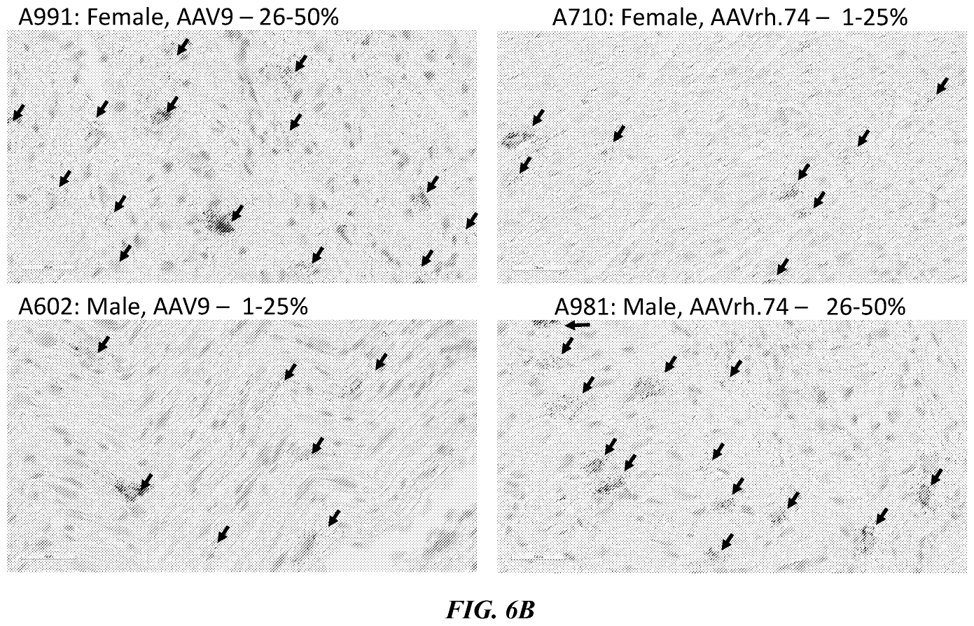

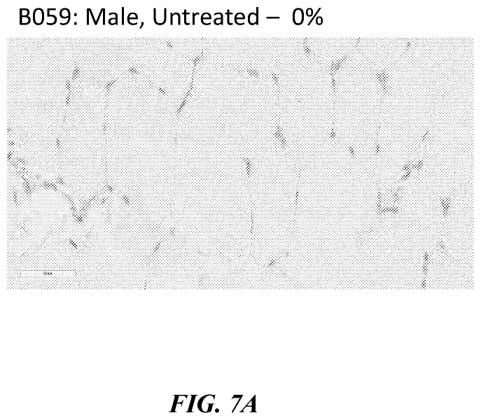

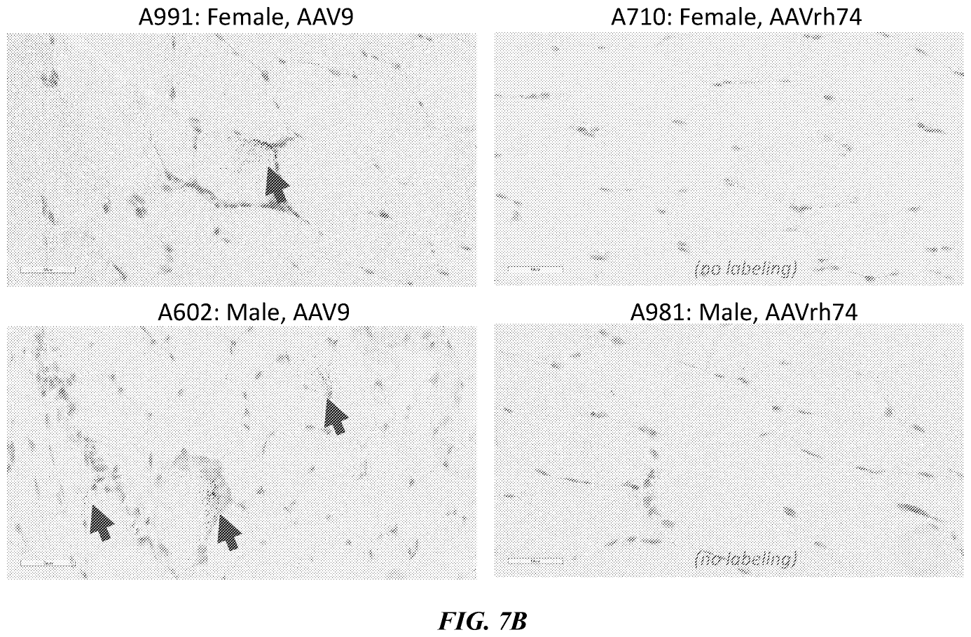

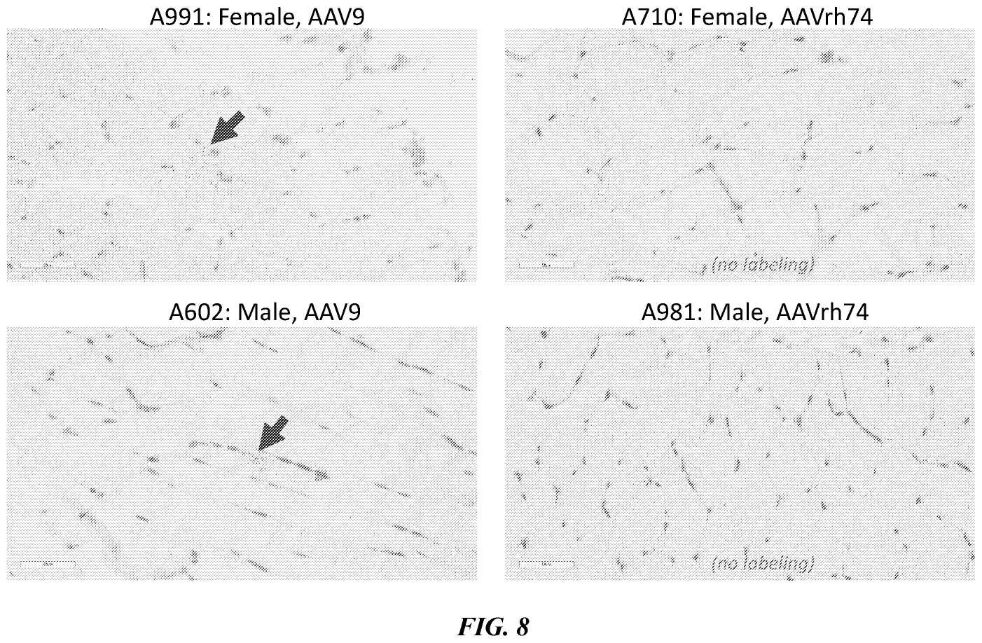

FIG. 6A shows a micrograph of semi-quantitative mRNA analysis by RNAscope in an untreated left ventricle. FIG. 6B shows micrographs of semi-quantitative mRNA analysis by RNAscope in treated left ventricles. FIG. 7A shows a micrograph of semi-quantitative mRNA analysis by RNAscope in an untreated quadricep. FIG. 7B shows micrographs of semi-quantitative mRNA analysis by RNAscope in treated quadriceps. FIG. 8 shows micrographs of semi-quantitative mRNA analysis by RNAscope in treated gastrocnemius. Results are summarized for vehicle control (Table 3A), AAV9 (Table 3A), and AAVrh74 (Table 3C).

TABLE 3A

| | | | | % Expressing |
|---|---|---|---|---|
| Animal | Tissue | Location | Treatment | Cells |
| B059 | Heart | left ventricle | Untreated, vehicle | 0% |
| B059 | Muscle | Quadricep | Untreated, vehicle | 0% |
| B059 | Liver | Left Lobe | Untreated, vehicle | 0% |

TABLE 3B

| | | | | % Expressing |
|---|---|---|---|---|
| Animal | Tissue | Location | Treatment | Cells |
| A991 | Heart | left ventricle | Treated, AAV9 | 26-50% |
| A991 | Heart | right ventricle | Treated, AAV9 | 26-50% |
| A991 | Heart | left atrium | Treated, AAV9 | 1-25% |
| A991 | Heart | right atrium | Treated, AAV9 | 26-50% |
| A991 | Muscle | quadricep | Treated, AAV9 | 0% |
| A991 | Muscle | gastrocnemius | Treated, AAV9 | 0% |
| A991 | Liver | left lobe | Treated, AAV9 | 26-50% |
| A991 | Liver | right lobe | Treated, AAV9 | 51-75% |
| A602 | Heart | left ventricle | Treated, AAV9 | 1-25% |
| A602 | Heart | right ventricle | Treated, AAV9 | 1-25% |
| A602 | Heart | left atrium | Treated, AAV9 | 26-50% |
| A602 | Heart | right atrium | Treated, AAV9 | 1-25% |
| A602 | Muscle | quadricep | Treated, AAV9 | 1-25% |
| A602 | Muscle | gastrocnemius | Treated, AAV9 | 1-25% |
| A602 | Liver | left lobe | Treated, AAV9 | 51-75% |
| A602 | Liver | right lobe | Treated, AAV9 | 51-75% |

TABLE 3C

| | | | | % Expressing |
|---|---|---|---|---|
| Animal | Tissue | Location | Treatment | Cells |
| A710 | Heart | left ventricle | Treated, AAVrh.74 | 1-25% |
| A710 | Heart | right ventricle | Treated, AAVrh.74 | 1-25% |
| A710 | Heart | left atrium | Treated, AAVrh.74 | 1-25% |
| A710 | Heart | right atrium | Treated, AAVrh.74 | 1-25% |
| A710 | Muscle | quadricep | Treated, AAVrh.74 | 0% |
| A710 | Muscle | gastrocnemius | Treated, AAVrh.74 | 1-25% |
| A710 | Liver | left lobe | Treated, AAVrh.74 | 51-75% |
| A710 | Liver | right lobe | Treated, AAVrh.74 | 51-75% |
| A981 | Heart | left ventricle | Treated, AAVrh.74 | 26-50% |
| A981 | Heart | right ventricle | Treated, AAVrh.74 | 26-50% |
| A981 | Heart | left atrium | Treated, AAVrh.74 | 0% |
| A981 | Heart | right atrium | Treated, AAVrh.74 | 26-50% |
| A981 | Muscle | quadricep | Treated, AAVrh.74 | 0% |
| A981 | Muscle | gastrocnemius | Treated, AAVrh.74 | 0% |
| A981 | Liver | left lobe | Treated, AAVrh.74 | 51-75% |
| A981 | Liver | right lobe | Treated, AAVrh.74 | 26-51% |

### LAMP2B Protein—ELISA

Approximately 125 mg of tissue was homogenized in 500 μL of lysis buffer using 0.9-2.00 mm stainless steel beads (Next Advance) and a Next Advance Bullet Blender 24. The lysis buffer contains 300 mM NaCl, 20 mM EDTA, 100 mM Tris pH 8.0, 2% NP-40 and 0.2% SDS with Complete™ EDTA-free protease inhibitor and PhosSTOP™ phosphatase inhibitor. Total protein was assessed by BCA (Thermo). 100 mg of total protein was loaded per well. A standard curve was constructed using purified human LAMP2 protein (Origene). ELISA was performed with a mouse monoclonal antibody (H4B4, Novus Biologicals) as the capture antibody, a goat polyclonal antibody (R&D Systems) as the detection antibody, HRP-linked antibody: Donkey anti-goat (Millipore) as the secondary antibody. Plates were developed with TMB (Thermo) and quantified on a spectrophotometer (Spectramax M5c).

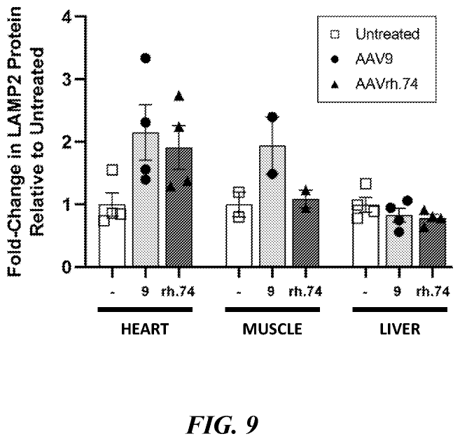

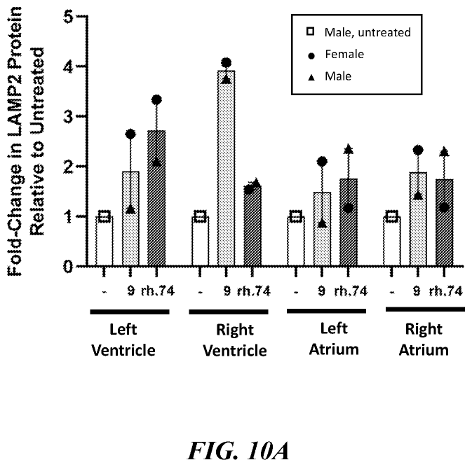

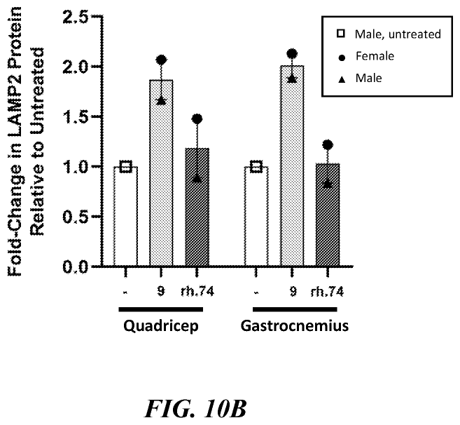

FIG. 9 shows a bar graph of protein quantification in tissues most affected in Danon disease by ELISA. FIG. 10A shows a bar graph of protein quantification in regions of the heart by ELISA. FIG. 10B shows a bar graph of protein quantification in muscles by ELISA.

### Clinical Pathology

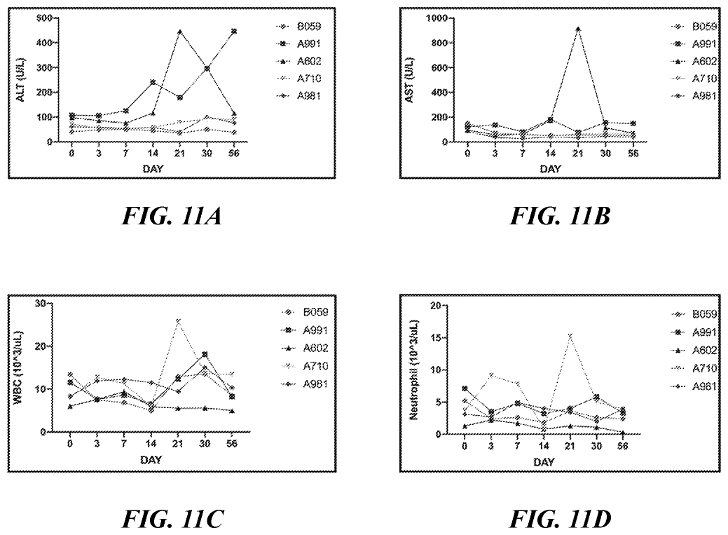

Pathological effects of the vectors were assessed. FIGS. 11A-11D show line graphs of clinical pathology measurement in NHP serum over course of study. Clinical pathology levels were assessed as changes in (FIG. 11A) alanine aminotransferase, ALT; (FIG. 11B) aspartate aminotransferase, AST; (FIG. 11C) white blood cells, WBC; and (FIG. 11D) neutrophils over the study duration. B059 is the vehicle control. A991 and A602 are AAV9-treated animals. A710 and A981 are AAVrh74-treated animals.

### CONCLUSIONS

AAV-based gene therapy using a LAMP2B transgene was well tolerated in non-human primates at vector dose 1.0×10¹³ vg/kg. This result is an important and unexpected result because experiments with AAV-based gene therapy for some other transgenes have demonstrated pathological effects at doses equal to or lower than 1.0×10¹³ vg/kg. Furthermore, both AAV9 and AAVrh74 were well tolerated. Elevated levels of certain markers were observed in A602 and A710 animals at day 21, but these outliers may be due to experimental error or pathology that was self-resolving.

Both vectors localized to and transduce target tissues for treatment of Danon disease (heart and muscle), but not as much in the brain or gonads. Expression in the gonads would be undesirable for safety reasons. As expected significant amounts of vector accumulate in the liver, which is desirable because liver is a tissue affected by Danon disease. Vector is present in each quadrant of the heart and in both quadricep and gastrocnemius muscles. This is a desirable result for treatment of Danon disease.

Localization of a serotype of AAV vector (e.g. AAV9) is not predictive of localization of others (e.g. AAVrh74). This experiment demonstrates that AAVrh74 achieves desirable localization for treatment of Danon disease, or other diseases with etiology linked to heart and muscle tissues. Both LAMP2B transgene mRNA and protein are expressed in the same sets of tissues in both AAV9 and AAVrh74 groups. Expression is comparable between vector serotypes. The number of animals in the study is too few to discern statistically significant trends in expression levels between AAV9 and AAVrh74. RNAscope suggests that a similar fraction of cells are infected in heart, muscle, and liver tissues in AAV9 and AAVrh74 groups.

These data demonstrate that AAVrh74 may be used as a vector to deliver LAMP2B to tissues relevant to treatment of Danon disease. AAVrh74 was non-inferior to AAV9 in these experiments.

What is claimed is:

1. A recombinant adeno-associated virus (rAAV) gene therapy vector, comprising a polynucleotide comprising a 5′ ITR, an expression cassette, and a 3′ ITR; and a capsid protein,
wherein the expression cassette comprises a transgene encoding a lysosome-associated membrane protein 2 (LAMP-2) or a functional variant thereof,
wherein the expression cassette is flanked by the 5′ ITR and the 3′ ITR, and
wherein the capsid protein comprises an AAVrh.74 capsid protein or a functional variant thereof.

2. The rAAV gene therapy vector of claim 1, wherein the LAMP-2 is selected from LAMP-2A, LAMP-2B and LAMP-2C.

3. The rAAV gene therapy vector of claim 1, wherein the capsid protein has at least 95% sequence identity to an amino acid sequence selected from SEQ ID NOs: 2-4.

4. The rAAV gene therapy vector of claim 3, wherein the capsid protein shares at least 95% sequence identity to SEQ ID NOs: 2.

5. The rAAV gene therapy vector of claim 4, wherein the capsid protein shares at least 97% sequence identity to SEQ ID NOs: 2.

6. The rAAV gene therapy vector of claim 5, wherein the capsid protein shares at least 99% sequence identity to SEQ ID NOs: 2.

7. The rAAV gene therapy vector of any one of claims 1-6, wherein the capsid protein is an AAVrh.74 capsid protein.

8. The rAAV gene therapy vector of any one of claims 1-7, wherein the 5′ ITR and the 3′ ITR are each respectively the 5′ ITR of AAV2 and the 3′ ITR of AAV2, or variants thereof.

9. The rAAV gene therapy vector of claim 8, wherein the 5′ ITR shares at least 98% identity to SEQ ID NO: 13 and the 3′ ITR shares at least 98% identity to SEQ ID NO: 14.

10. The rAAV gene therapy vector of any one of claims 1-9, wherein the transgene is codon-optimized for expression in a human host cell.

11. The rAAV gene therapy vector of any one of claims 1-10, wherein the expression cassette contains fewer CpG sites than SEQ ID: 6.

12. The rAAV gene therapy vector of any one of claims 1-11, wherein the expression cassette contains fewer cryptic splice sites than SEQ ID: 6.

13. The rAAV gene therapy vector of any one of claims 1-12, wherein the expression cassette encodes fewer alternative open reading frames than SEQ ID: 6.

14. The rAAV gene therapy vector of any one of claims 1 to 13, wherein the transgene shares at least 95% identity to a sequence selected from SEQ ID NO: 7-9.

15. The rAAV gene therapy vector of claim 14, wherein the transgene shares at least 99% identity to a sequence selected from SEQ ID NO: 7-9.

16. The rAAV gene therapy vector of claim 15, wherein the transgene comprises a sequence selected from SEQ ID NO: 7-9.

17. The rAAV gene therapy vector of any one of claims 1 to 16, where the expression cassette comprises a consensus optimal Kozak sequence operatively linked to the transgene, wherein the consensus optimal Kozak sequence comprises SEQ ID NO: 20.

18. The rAAV gene therapy vector of any one of claims 1 to 17, where the expression cassette comprises a full-length polyA sequence operatively linked to the transgene, wherein the full-length polyA sequence comprises SEQ ID NO: 26.

19. The rAAV gene therapy vector of any one of claims 1 to 18, where the expression cassette comprises no start codon 5′ to the start codon of the transgene.

20. The rAAV gene therapy vector of any one of claims 1 to 19, wherein the expression cassette comprises operatively linked, in the 5′ to 3′ direction, a first inverse terminal repeat, an enhancer/promoter region, an intron, a consensus optimal Kozak sequence, the transgene, a 3′ untranslated region including a full-length polyA sequence, and a second inverse terminal repeat, where the expression cassette comprises no start codon 5′ to the start codon of the transgene.

21. The rAAV gene therapy vector of claim 20, wherein the enhancer/promoter region comprises in the 5′ to 3′ direction a CMV IE Enhancer and a Chicken Beta-Actin Promoter, and optionally wherein the enhancer/promoter region further comprises a first exon and first intron of a chicken beta-actin gene and a splice acceptor of a rabbit beta-globin gene.

22. The rAAV gene therapy vector of claim 20, wherein the enhancer/promoter region comprises a tissues-specific promoter capable of mediating increased expression in cardiac tissue and/or skeletal muscle tissue compared to liver tissue.

23. The rAAV gene therapy vector of any one of claims 1 to 21, wherein the expression cassette shares at least 95% identity to a sequence selected from SEQ ID NOs: 10-12.

24. The rAAV gene therapy vector of claim 23, wherein the expression cassette comprises a sequence selected from SEQ ID NOs: 10-12.

25. A pharmaceutical composition comprising the rAAV gene therapy vector of any one of claims 1 to 24.

26. A method of treating or preventing Danon disease or another autophagy disorder in a subject in need thereof, comprising administering to the subject the rAAV gene therapy vector of any one of claims 1 to 24 or the pharmaceutical composition of claim 25.

27. The method of claim 26, wherein the rAAV gene therapy vector or pharmaceutical composition is administered via a route selected from the group consisting of intravenous, intra-arterial, intracardiac, intracoronary, intramyocardial, intrarenal, intraurethral, epidural, and intramuscular.

28. The method of claim 26 or claim 27, wherein the autophagy disorder is selected from the group consisting of end-stage heart failure, myocardial infarction, drug toxicities, diabetes, end-stage renal failure, and aging.

29. The method of any one of claims 26 to 28, wherein the subject is a human.

30. The method of any one of claims 26 to 29, wherein the subject is exhibiting symptoms of Danon disease or another autophagy disorder.

31. The method of any one of claims 26 to 30, wherein the subject has been identified as having reduced or non-detectable expression of endogenous LAMP-2.

32. The method of any one of claims 26 to 31, wherein the subject has been identified as having a mutated LAMP-2 gene.

33. The method of any one of claims 26 to 32, wherein the rAAV gene therapy vector is administered at a dose of about 3×10¹² vg/kg to about 3×10¹⁴ vg/kg.

34. The method of any one of claims 26 to 33, wherein the rAAV gene therapy vector is administered at a dose of about 3×10¹² vg/kg to about 1.2×10¹³ vg/kg.

35. The method of any one of claims 26 to 33, wherein the rAAV gene therapy vector is administered at a dose of about 1.0×10¹³ vg/kg.

36. The method of any one of claims 26 to 35, wherein the dose of rAAV gene therapy vector does not cause clinical pathology when administered, optionally when administered at a dose of about 1.0×10¹³ vg/kg.

37. The method of any one of claims 26 to 36, wherein administration of the rAAV gene therapy vector transduces one or more of heart, muscle, and liver.

38. The method of any one of claims 26 to 37, wherein administration of the rAAV gene therapy vector causes LAMP2B mRNA expression in one or more of heart, muscle, and liver.

39. The method of any one of claims 26 to 38, wherein administration of the rAAV gene therapy vector causes LAMP2B protein expression in one or more of heart, muscle, and liver.

40. The method of any one of claims 26 to 39, wherein administration of the rAAV gene therapy vector causes infection with the rAAV gene therapy vector of at least about 10%, at least about 20%, or at least about 30% of cells in one or more of heart, muscle, and liver.

41. The method of any one of claims 26 to 40, wherein administration of the rAAV gene therapy vector causes transduction of the rAAV gene therapy vector in gonads at less 0.1 vector genomes (vg) per diploid genome.

42. The method of any one of claims 26 to 41, wherein administration of the rAAV gene therapy vector causes LAMP2B mRNA expression in gonads at less than 2×10⁴ mRNA copies per μg total RNA.

43. The method of any one of claims 26 to 42, wherein administration of the rAAV gene therapy vector causes no LAMP2B protein expression in brain and/or gonads.

44. The method of any one of claims 26 to 43, wherein administration of the rAAV gene therapy vector transduces and/or causes transgene expression at about the same level as an AAV9 gene therapy vector having the same expression cassette.

45. A method of delivering a LAMP-2 polynucleotide encoding a LAMP-2 protein to a cell, comprising contacting the cell with the rAAV gene therapy vector of any one of claims 1 to 24 or the pharmaceutical composition of claim 25, wherein the cell is optionally selected from a heart cell, a lung cell, and/or a muscle cell.

46. A method of transducing cells, comprising contacting the cells with the rAAV gene therapy vector of any one of claims 1 to 24 or the pharmaceutical composition of claim 25, wherein the cell is optionally selected from a heart cell, a lung cell, and/or a muscle cell.

47. A method of delivering a LAMP-2 polynucleotide encoding a LAMP-2 protein to a tissue and/or expressing a LAMP-2 protein in a tissue, comprising contacting the tissue with the rAAV gene therapy vector of any one of claims 1 to 24 or the pharmaceutical composition of claim 25, wherein the tissue is optionally selected from heart tissue, lung tissue, and/or muscle tissue.

48. A method of delivering a LAMP-2 polynucleotide encoding a LAMP-2 protein to a subject and/or expressing a LAMP-2 protein in a subject, comprising administering to the subject the rAAV gene therapy vector of any one of claims 1 to 24 or the pharmaceutical composition of claim 25.

49. The method of claim 48, wherein the rAAV gene therapy vector or pharmaceutical composition is administered via a route selected from the group consisting of intravenous, intra-arterial, intracardiac, intracoronary, intramyocardial, intrarenal, intraurethral, epidural, and intramuscular.

50. The method of claim 48 or claim 49, wherein the subject suffers from or is at risk for an autophagy disorder selected from the group consisting of Danon disease, end-stage heart failure, myocardial infarction, drug toxicities, diabetes, end-stage renal failure, and aging.

51. The method of any one of claims 48 to 50, wherein the subject is a human.

52. The method of any one of claims 48 to 51, wherein the subject is exhibiting symptoms of the autophagy disorder.

53. The method of any one of claims 48 to 52, wherein the subject has been identified as having reduced or non-detectable expression of endogenous LAMP-2.

54. The method of any one of claims 48 to 53, wherein the subject has been identified as having a mutated LAMP-2 gene.

55. The method of any one of claims 48 to 54, wherein the rAAV gene therapy vector is administered at a dose of about 3×10¹² vg/kg to about 3×10¹⁴ vg/kg.

56. The method of any one of claims 48 to 55, wherein the rAAV gene therapy vector is administered at a dose of about 3×10¹² vg/kg to about 1.2×10¹³ vg/kg.

57. The method of any one of claims 48 to 56, wherein the rAAV gene therapy vector is administered at a dose of about 1.0×10¹³ vg/kg.

58. The method of any one of claims 48 to 57, wherein the dose of rAAV gene therapy vector does not cause clinical pathology when administered, optionally when administered at a dose of about 1.0×10¹³ vg/kg.

59. The method of any one of claims 48 to 58, wherein administration of the rAAV gene therapy vector transduces one or more of heart, muscle, and liver.

60. The method of any one of claims 48 to 59, wherein administration of the rAAV gene therapy vector causes LAMP2B mRNA expression in one or more of heart, muscle, and liver.

61. The method of any one of claims 48 to 60, wherein administration of the rAAV gene therapy vector causes LAMP2B protein expression in one or more of heart, muscle, and liver.

62. The method of any one of claims 48 to 61, wherein administration of the rAAV gene therapy vector causes infection with the rAAV gene therapy vector of at least about 10%, at least about 20%, or at least about 30% of cells in one or more of heart, muscle, and liver.

63. The method of any one of claims 48 to 62, wherein administration of the rAAV gene therapy vector causes transduction of the rAAV gene therapy vector in gonads at less 0.1 vector genomes (vg) per diploid genome.

64. The method of any one of claims 48 to 63, wherein administration of the rAAV gene therapy vector causes LAMP2B mRNA expression in gonads at less than 2×10⁴ mRNA copies per μg total RNA.

65. The method of any one of claims 48 to 64, wherein administration of the rAAV gene therapy vector causes no LAMP2B protein expression in brain and/or gonads.

66. The method of any one of claims 48 to 65, wherein administration of the rAAV gene therapy vector transduces and/or causes transgene expression at about the same level as an AAV9 gene therapy vector having the same expression cassette.


SEQUENCE LISTING (35 sequences)

SEQ ID NO: 1 - DNA, 2217 bp, Non-human primate Adeno-Associated Virus
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcttttgggggcaacctcgggcgcgcagtcttccaggccaaaaagcgggttctcgaacctctgggcctggttgaatcgccggttaagacggctcctggaaagaagagaccggtagagccatcaccccagcgctctccagactcctctacgggcatcggcaagaaaggccagcagcccgcaaaaaagagactcaattttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgcacctgggccctgcccacctacaacaaccacctctacaagcaaatctccaacgggacctcgggaggaagcaccaacgacaacacctacttcggctacagcaccccctgggggtattttgacttcaacagattccactgccacttttcaccacgtgactggcagcgactcatcaacaacaactggggattccggcccaagaggctcaacttcaagctcttcaacatccaagtcaaggaggtcacgcagaatgaaggcaccaagaccatcgccaataaccttaccagcacgattcaggtctttacggactcggaataccagctcccgtacgtgctcggctcggcgcaccagggctgcctgcctccgttcccggcggacgtcttcatgattcctcagtacgggtacctgactctgaacaatggcagtcaggctgtgggccggtcgtccttctactgcctggagtactttccttctcaaatgctgagaacgggcaacaactttgaattcagctacaacttcgaggacgtgcccttccacagcagctacgcgcacagccagagcctggaccggctgatgaaccctctcatcgaccagtacttgtactacctgtcccggactcaaagcacgggcggtactgcaggaactcagcagttgctattttctcaggccgggcctaacaacatgtcggctcaggccaagaactggctacccggtccctgctaccggcagcaacgcgtctccacgacactgtcgcagaacaacaacagcaactttgcctggacgggtgccaccaagtatcatctgaatggcagagactctctggtgaatcctggcgttgccatggctacccacaaggacgacgaagagcgattttttccatccagcggagtcttaatgtttgggaaacagggagctggaaaagacaacgtggactatagcagcgtgatgctaaccagcgaggaagaaataaagaccaccaacccagtggccacagaacagtacggcgtggtggccgataacctgcaacagcaaaacgccgctcctattgtaggggccgtcaatagtcaaggagccttacctggcatggtgtggcagaaccgggacgtgtacctgcagggtcccatctgggccaagattcctcatacggacggcaactttcatccctcgccgctgatgggaggctttggactgaagcatccgcctcctcagatcctgattaaaaacacacctgttcccgcggatcctccgaccaccttcaatcaggccaagctggcttctttcatcacgcagtacagtaccggccaggtcagcgtggagatcgagtgggagctgcagaaggagaacagcaaacgctggaacccagagattcagtacacttccaactactacaaatctacaaatgtggactttgctgtcaatactgagggtacttattccgagcctcgccccattggcacccgttacctcacccgtaatctgtaa

SEQ ID NO: 2 - protein, 535 aa, Non-human primate adeno-associated Virus
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 3 - protein, 398 aa, Non-human primate adeno-associated virus
STIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 4 - protein, 332 aa, Non-human primate adeno-associated virus
RTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 5 - protein, 409 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQECSLDDDTILIPIIVGAGLSGLIIVIVIAYVIGRRKSYAGYQT

SEQ ID NO: 6 - DNA, 1233 bp, Homo sapiens
atggtgtgcttccgcctcttcccggttccgggctcagggctcgttctggtctgcctagtcctgggagctgtgcggtcttatgcattggaacttaatttgacagattcagaaaatgccacttgcctttatgcaaaatggcagatgaatttcacagttcgctatgaaactacaaataaaacttataaaactgtaaccatttcagaccatggcactgtgacatataatggaagcatttgtggggatgatcagaatggtcccaaaatagcagtgcagttcggacctggcttttcctggattgcgaattttaccaaggcagcatctacttattcaattgacagcgtctcattttcctacaacactggtgataacacaacatttcctgatgctgaagataaaggaattcttactgttgatgaacttttggccatcagaattccattgaatgacctttttagatgcaatagtttatcaactttggaaaagaatgatgttgtccaacactactgggatgttcttgtacaagcttttgtccaaaatggcacagtgagcacaaatgagttcctgtgtgataaagacaaaacttcaacagtggcacccaccatacacaccactgtgccatctcctactacaacacctactccaaaggaaaaaccagaagctggaacctattcagttaataatggcaatgatacttgtctgctggctaccatggggctgcagctgaacatcactcaggataaggttgcttcagttattaacatcaaccccaatacaactcactccacaggcagctgccgttctcacactgctctacttagactcaatagcagcaccattaagtatctagactttgtctttgctgtgaaaaatgaaaaccgattttatctgaaggaagtgaacatcagcatgtatttggttaatggctccgttttcagcattgcaaataacaatctcagctactgggatgcccccctgggaagttcttatatgtgcaacaaagagcagactgtttcagtgtctggagcatttcagataaatacctttgatctaagggttcagcctttcaatgtgacacaaggaaagtattctacagcccaagagtgttcgctggatgatgacaccattctaatcccaattatagttggtgctggtctttcaggcttgattatcgttatagtgattgcttacgtaattggcagaagaaaaagttatgctggatatcagactctgtaa

SEQ ID NO: 7 - DNA, 1233 bp, Artificial Sequence
  Made in Lab - Engineered Lamp-2B coding sequence
atggtctgcttcagactgttccctgtccctggatctggtctggtgcttgtgtgcttggtgctgggtgctgtgagatcctatgcccttgagctgaacctgactgactcagaaaatgccacttgcctgtatgccaagtggcagatgaacttcactgtgagatatgagactaccaacaagacctacaagactgtgaccatctcagaccatggcactgtcacctacaatggatcaatctgtggtgatgatcagaatggcccaaagatagcagtgcagtttgggcccggtttttcctggattgctaacttcaccaaggcagcctccacctacagcattgactcagtcagcttcagctacaacactggggataacaccaccttccctgacgcagaggacaagggaatccttactgtggacgaactcctggcaatcagaatcccccttaacgacctgttcagatgcaactccctttcaacccttgaaaagaatgatgtggtgcaacactattgggacgtcctggtgcaagcctttgtgcagaatgggacagtgagtaccaacgagttcctctgtgacaaggacaagaccagcactgtggcccccactatccacaccactgtgcccagccctaccactacccccacccctaaagagaagccagaagctggaacctactcagtcaacaatggaaatgacacatgcctccttgccaccatgggactgcagctgaacatcactcaggacaaggtggcctcagtgattaacatcaaccctaacaccactcatagcactgggagctgcagatcacatacagctctgctgaggctcaactcctccaccatcaagtacctggactttgtgtttgctgtgaagaatgagaacaggttctacctcaaggaagtgaacatttccatgtacctggtcaatggttcagtgttctctattgccaacaacaatctgagctactgggatgcacccctgggatcctcctacatgtgcaacaaggagcagactgtgagtgtgtcaggtgcttttcagatcaacacttttgacctgagggtgcagcccttcaatgtgactcagggaaagtactccactgcacaagagtgttccttggatgatgacactatcctcatccccattattgtgggagctggactgtcaggattgattatagtgattgtgattgcttatgtgattggaaggagaaagagctatgctggctaccagaccctgtaa

SEQ ID NO: 8 - DNA, 1233 bp, Artificial Sequence
  Made in Lab - Engineered Lamp-2B coding sequence
atggtgtgctttagactgtttcctgtgcctggttcagggctggtcctggtctgtctggtgctgggggctgtcagaagctatgccttggagctgaacctcactgatagtgaaaatgccacttgtctgtatgctaagtggcagatgaacttcactgtgagatatgaaaccaccaacaagacttacaaaacagtgaccatctcagatcatggaactgtgacctacaacggcagcatttgtggagacgaccagaacggaccaaaaatcgctgtccaatttgggcctggattctcctggattgccaatttcactaaagctgcctccacatattcaattgactcagtgtccttctcctacaacactggggacaacactactttccctgatgctgaagataagggaatcttgacagtggatgagctgctggctatcaggatccctttgaatgacctgtttaggtgtaattcactgagcactctggagaagaacgacgtggtgcagcactactgggacgtgctggtgcaggcctttgtgcagaacggcactgtgtccaccaacgaattcctgtgtgataaggacaaaacttccactgtggcacctacaattcacactactgtgccttcacctaccaccactccaactccaaaggaaaagcctgaagcaggaacctactctgtgaacaatggcaatgatacctgtctgttggccaccatgggcctccaactgaacattactcaggacaaggtggcctcagtgattaacattaaccccaacactacccactccactggcagctgtagatcacacacagccttgctcagactgaatagcagcaccatcaagtatttggattttgtgtttgcagtgaagaatgaaaacaggttctacctgaaggaagtcaacatctcaatgtacctggtgaacggctcagtgttcagcattgccaacaacaacctctcctattgggacgctccactggggagcagctacatgtgtaacaaggaacagactgtgtcagtgtcaggagccttccagattaacacctttgatctgagggtccaaccctttaatgtcactcaaggaaagtatagcactgcccaggagtgctccctggatgatgacaccattctgattccaatcattgtgggtgcaggactttctgggcttattattgtgattgtgattgcctatgtgattggcagaaggaaatcctatgcagggtaccaaactctgtaa

SEQ ID NO: 9 - DNA, 1233 bp, Artificial Sequence
  Made in Lab - Engineered Lamp-2B coding sequence
atggtctgttttaggctgttccctgtccctggttcaggactggtcttagtgtgtctggtgcttggagctgtcagaagctatgccctggagctgaacctgactgactcagaaaatgccacttgcctgtatgccaagtggcagatgaacttcactgtcagatatgaaaccaccaacaagacctataagactgtgaccatctcagaccatggcactgtgacttacaatgggtcaatttgtggagatgaccagaatggccctaagatagctgtccagtttggtccaggattcagctggattgccaacttcaccaaggcagccagcacctacagcattgactctgtgtccttctcctacaacacaggagacaacaccactttccctgatgcagaggacaaaggtatcctgactgtggatgagttgctggcaatcaggatcccactgaacgatctgttcaggtgcaactcactgtccactctggaaaagaatgatgtggtgcagcactattgggatgtgctagtccaggcctttgtccagaatgggactgtgtcaactaatgagttcctgtgtgacaaggacaagacaagcactgtagcccccactatccataccacagtacctagccccaccactactccaacccccaaggagaagcctgaggctggcacctactcagtgaacaatgggaatgacacctgtttgctggccactatgggactccaactgaacatcacccaggacaaagtggcctctgtgatcaatatcaatcccaacaccacccacagcactgggtcctgcagaagccacactgccctcctgaggctcaactcatcaactatcaagtacttggattttgtgtttgcagtgaagaatgagaacagattctacctcaaagaggtcaacatttcaatgtacctggtgaatgggagtgtgttctccattgctaacaacaacctgagctactgggatgcccctctgggctcctcatacatgtgcaacaaggaacagactgtgagtgtgtcaggggccttccagatcaacacttttgacctgagagtgcagccctttaatgtgacacagggaaagtacagcactgctcaggagtgcagcctggatgatgacactatcctgatccctatcattgtgggggcaggcctgtctggactcattattgtgattgtgattgcctatgtgatagggagaaggaagtcttatgctggataccagaccctgtaa

SEQ ID NO: 10 - DNA, 4549 bp, Artificial Sequence
  Made in Lab - Engineered expression cassette
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagcggccgccagccgccaccatggtctgcttcagactgttccctgtccctggatctggtctggtgcttgtgtgcttggtgctgggtgctgtgagatcctatgcccttgagctgaacctgactgactcagaaaatgccacttgcctgtatgccaagtggcagatgaacttcactgtgagatatgagactaccaacaagacctacaagactgtgaccatctcagaccatggcactgtcacctacaatggatcaatctgtggtgatgatcagaatggcccaaagatagcagtgcagtttgggcccggtttttcctggattgctaacttcaccaaggcagcctccacctacagcattgactcagtcagcttcagctacaacactggggataacaccaccttccctgacgcagaggacaagggaatccttactgtggacgaactcctggcaatcagaatcccccttaacgacctgttcagatgcaactccctttcaacccttgaaaagaatgatgtggtgcaacactattgggacgtcctggtgcaagcctttgtgcagaatgggacagtgagtaccaacgagttcctctgtgacaaggacaagaccagcactgtggcccccactatccacaccactgtgcccagccctaccactacccccacccctaaagagaagccagaagctggaacctactcagtcaacaatggaaatgacacatgcctccttgccaccatgggactgcagctgaacatcactcaggacaaggtggcctcagtgattaacatcaaccctaacaccactcatagcactgggagctgcagatcacatacagctctgctgaggctcaactcctccaccatcaagtacctggactttgtgtttgctgtgaagaatgagaacaggttctacctcaaggaagtgaacatttccatgtacctggtcaatggttcagtgttctctattgccaacaacaatctgagctactgggatgcacccctgggatcctcctacatgtgcaacaaggagcagactgtgagtgtgtcaggtgcttttcagatcaacacttttgacctgagggtgcagcccttcaatgtgactcagggaaagtactccactgcacaagagtgttccttggatgatgacactatcctcatccccattattgtgggagctggactgtcaggattgattatagtgattgtgattgcttatgtgattggaaggagaaagagctatgctggctaccagaccctgtaaaagggcgaattccagcacacgcgtcctaggagctcgagtactactggcggccgttactagtggatccgcggtacaagtaagcatgcaagcttcgaggacggggtgaactacgcctgaatcaagcttatcgataaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctcgataccgtcgatatcgatcctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgaagcaattcgttgatctgaatttcgaccacccataatagatctcccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 11 - DNA, 4549 bp, Artificial Sequence
  Made in Lab - Engineered expression cassette
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagcggccgccagccgccaccatggtgtgctttagactgtttcctgtgcctggttcagggctggtcctggtctgtctggtgctgggggctgtcagaagctatgccttggagctgaacctcactgatagtgaaaatgccacttgtctgtatgctaagtggcagatgaacttcactgtgagatatgaaaccaccaacaagacttacaaaacagtgaccatctcagatcatggaactgtgacctacaacggcagcatttgtggagacgaccagaacggaccaaaaatcgctgtccaatttgggcctggattctcctggattgccaatttcactaaagctgcctccacatattcaattgactcagtgtccttctcctacaacactggggacaacactactttccctgatgctgaagataagggaatcttgacagtggatgagctgctggctatcaggatccctttgaatgacctgtttaggtgtaattcactgagcactctggagaagaacgacgtggtgcagcactactgggacgtgctggtgcaggcctttgtgcagaacggcactgtgtccaccaacgaattcctgtgtgataaggacaaaacttccactgtggcacctacaattcacactactgtgccttcacctaccaccactccaactccaaaggaaaagcctgaagcaggaacctactctgtgaacaatggcaatgatacctgtctgttggccaccatgggcctccaactgaacattactcaggacaaggtggcctcagtgattaacattaaccccaacactacccactccactggcagctgtagatcacacacagccttgctcagactgaatagcagcaccatcaagtatttggattttgtgtttgcagtgaagaatgaaaacaggttctacctgaaggaagtcaacatctcaatgtacctggtgaacggctcagtgttcagcattgccaacaacaacctctcctattgggacgctccactggggagcagctacatgtgtaacaaggaacagactgtgtcagtgtcaggagccttccagattaacacctttgatctgagggtccaaccctttaatgtcactcaaggaaagtatagcactgcccaggagtgctccctggatgatgacaccattctgattccaatcattgtgggtgcaggactttctgggcttattattgtgattgtgattgcctatgtgattggcagaaggaaatcctatgcagggtaccaaactctgtaaaagggcgaattccagcacacgcgtcctaggagctcgagtactactggcggccgttactagtggatccgcggtacaagtaagcatgcaagcttcgaggacggggtgaactacgcctgaatcaagcttatcgataaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctcgataccgtcgatatcgatcctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgaagcaattcgttgatctgaatttcgaccacccataatagatctcccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 12 - DNA, 4549 bp, Artificial Sequence
  Made in Lab - Engineered expression cassette
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagcggccgccagccgccaccatggtctgttttaggctgttccctgtccctggttcaggactggtcttagtgtgtctggtgcttggagctgtcagaagctatgccctggagctgaacctgactgactcagaaaatgccacttgcctgtatgccaagtggcagatgaacttcactgtcagatatgaaaccaccaacaagacctataagactgtgaccatctcagaccatggcactgtgacttacaatgggtcaatttgtggagatgaccagaatggccctaagatagctgtccagtttggtccaggattcagctggattgccaacttcaccaaggcagccagcacctacagcattgactctgtgtccttctcctacaacacaggagacaacaccactttccctgatgcagaggacaaaggtatcctgactgtggatgagttgctggcaatcaggatcccactgaacgatctgttcaggtgcaactcactgtccactctggaaaagaatgatgtggtgcagcactattgggatgtgctagtccaggcctttgtccagaatgggactgtgtcaactaatgagttcctgtgtgacaaggacaagacaagcactgtagcccccactatccataccacagtacctagccccaccactactccaacccccaaggagaagcctgaggctggcacctactcagtgaacaatgggaatgacacctgtttgctggccactatgggactccaactgaacatcacccaggacaaagtggcctctgtgatcaatatcaatcccaacaccacccacagcactgggtcctgcagaagccacactgccctcctgaggctcaactcatcaactatcaagtacttggattttgtgtttgcagtgaagaatgagaacagattctacctcaaagaggtcaacatttcaatgtacctggtgaatgggagtgtgttctccattgctaacaacaacctgagctactgggatgcccctctgggctcctcatacatgtgcaacaaggaacagactgtgagtgtgtcaggggccttccagatcaacacttttgacctgagagtgcagccctttaatgtgacacagggaaagtacagcactgctcaggagtgcagcctggatgatgacactatcctgatccctatcattgtgggggcaggcctgtctggactcattattgtgattgtgattgcctatgtgatagggagaaggaagtcttatgctggataccagaccctgtaaaagggcgaattccagcacacgcgtcctaggagctcgagtactactggcggccgttactagtggatccgcggtacaagtaagcatgcaagcttcgaggacggggtgaactacgcctgaatcaagcttatcgataaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctcgataccgtcgatatcgatcctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcgaagcaattcgttgatctgaatttcgaccacccataatagatctcccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 13 - DNA, 130 bp, Adeno-associated viurs
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 14 - DNA, 130 bp, Adeno-associated virus
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 15 - protein, 410 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQDCSADDDNFLVPIAVGAALAGVLILVLLAYFIGLKHHHAGYEQF

SEQ ID NO: 16 - protein, 410 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAQECSLDDDTILIPIIVGAGLSGLIIVIVIAYVIGRRKSYAGYQTL

SEQ ID NO: 17 - protein, 411 aa, Homo sapiens
MVCFRLFPVPGSGLVLVCLVLGAVRSYALELNLTDSENATCLYAKWQMNFTVRYETTNKTYKTVTISDHGTVTYNGSICGDDQNGPKIAVQFGPGFSWIANFTKAASTYSIDSVSFSYNTGDNTTFPDAEDKGILTVDELLAIRIPLNDLFRCNSLSTLEKNDVVQHYWDVLVQAFVQNGTVSTNEFLCDKDKTSTVAPTIHTTVPSPTTTPTPKEKPEAGTYSVNNGNDTCLLATMGLQLNITQDKVASVININPNTTHSTGSCRSHTALLRLNSSTIKYLDFVFAVKNENRFYLKEVNISMYLVNGSVFSIANNNLSYWDAPLGSSYMCNKEQTVSVSGAFQINTFDLRVQPFNVTQGKYSTAEECSADSDLNFLIPVAVGVALGFLIIVVFISYMIGRRKSRTGYQSV

SEQ ID NO: 18 - DNA, 1730 bp, Artificial Sequence
  Made in Lab - promoter sequence
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaa

SEQ ID NO: 19 - DNA, 596 bp, Woodchuck hepatitis virus
attcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcc

SEQ ID NO: 20 - DNA, 13 bp, Artificial Sequence
  Kozak consensus sequence
gccgccaccatgg

SEQ ID NO: 21 - RNA, 13 nt, Artificial Sequence
  Kozak consensus sequence
gccgccrccaugg

SEQ ID NO: 22 - RNA, 8 nt, Artificial Sequence
  Kozak consensus sequence
  misc_feature (3)..(4): n is a, c, g, or u
  misc_feature (8)..(8): n is a, c, g, or u
agnnaugn

SEQ ID NO: 23 - RNA, 7 nt, Artificial Sequence
  Kozak consensus sequence
  misc_feature (2)..(3): n is a, c, g, or u
annaugg

SEQ ID NO: 24 - RNA, 7 nt, Artificial Sequence
  Kozak consensus sequence
accaugg

SEQ ID NO: 25 - RNA, 10 nt, Artificial Sequence
  Kozak consensus sequence
gacaccaugg

SEQ ID NO: 26 - DNA, 387 bp, Mus musculus
tggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatc

SEQ ID NO: 27 - DNA, 235 bp, Bos taurus
tcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaggacaatagcaggcatgctggggatgcggtgggctctatggcttctg

SEQ ID NO: 28 - DNA, 222 bp, Simian viurs 40
cagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggta

SEQ ID NO: 29 - DNA, 202 bp, Homo sapiens
ctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgggaagaaacctgtagggcctgc

SEQ ID NO: 30 - DNA, 21 bp, Artificial Sequence
  Primer
atcatgctattgcttcccgta

SEQ ID NO: 31 - DNA, 20 bp, Artificial Sequence
  Primer
gggccacaactcctcataaa

SEQ ID NO: 32 - DNA, 29 bp, Artificial Sequence
  Primer
cctccttgtataaatcctggttgctgtct

SEQ ID NO: 33 - DNA, 21 bp, Artificial Sequence
  Primer
atcatgctattgcttcccgta

SEQ ID NO: 34 - DNA, 20 bp, Artificial Sequence
  Primer
gggccacaactcctcataaa

SEQ ID NO: 35 - DNA, 29 bp, Artificial Sequence
  Primer
cctccttgtataaatcctggttgctgtct